(19) United States
(12) Patent Application Publication
(10) Pub. No.: US 2023/0212528 A1
(43) Pub. Date: Jul. 6, 2023

(54) MATERIALS AND METHODS FOR VIRAL PURIFICATION

(71) Applicant: Janssen Biotech, Inc., Horsham, PA (US)

(72) Inventors: Brian E. Tomkowicz, North Wales, PA (US); Matthew P. Ercolino, Stowe, PA (US); Stephen T. Spagnol, Conshohocken, PA (US); Sakya Sing Mohapatra, Conshohocken, PA (US); William Perry, Warrington, PA (US)

(21) Appl. No.: 18/000,525

(22) PCT Filed: Jun. 1, 2021
(86) PCT No.: PCT/US2021/035113

§ 371 Date: Dec. 2, 2022

Related U.S. Application Data
(60) Provisional application No. 63/033,449, filed on Jun. 2, 2020.
(60) Provisional application No. 63/033,492, filed on Jun. 2, 2020.
(60) Provisional application No. 63/033,531, filed on Jun. 2, 2020.
(60) Provisional application No. 63/033,549, filed on Jun. 2, 2020.
(60) Provisional application No. 63/033,631, filed on Jun. 2, 2020.
(60) Provisional application No. 63/033,643, filed on Jun. 2, 2020.
(60) Provisional application No. 63/033,731, filed on Jun. 2, 2020.

(51) Int. Cl. C12N 7/00; C12N 9/16

(52) U.S. Cl. CPC C12N 7/00; C12N 9/16; C12Y 301/31001; C12N 2750/14123; C12N 2750/14151

(57) ABSTRACT

Methods of purifying AAV particles using a chromatin-DNA nuclease (e.g., MNase), and compositions that include AAV particles and a chromatin-DNA nuclease (e.g., MNase) are described. Compositions and kits that include a chromatin-DNA nuclease (e.g., MNase) for the purification of AAV particles are also provided.

### CROSS-REFERENCE TO RELATED APPLICATIONS

This application is a National Stage application under 35 U.S.C. § of International Application No. PCT/US2021/035113 having an International Filing Date of Jun. 1, 2021, which claims the benefit of priority to U.S. Ser. No. 63/033,449 filed Jun. 2, 2020, U.S. Ser. No. 63/033,492 filed Jun. 2, 2020, U.S. Ser. No. 63/033,531 filed Jun. 2, 2020, U.S. Ser. No. 63/033,549 filed Jun. 2, 2020, U.S. Ser. No. 63/033,631 filed Jun. 2, 2020, U.S. Ser. No. 63/033,643 filed Jun. 2, 2020, and U.S. Ser. No. 63/033,731 filed Jun. 2, 2020, each of which is herein incorporated by reference in its entirety.

### BACKGROUND

Adeno-associated virus (AAV) is a non-enveloped virus that can be engineered to deliver nucleic acids to target cells, and has emerged as a useful vehicle in gene therapy and gene delivery applications. Recombinant AAV (rAAV), which lacks viral DNA, is essentially a protein-based nanoparticle engineered to traverse the cell membrane, where it can ultimately traffic and deliver its nucleic acid cargo into the nucleus of a cell. The properties conferred by this virus: sustained gene expression, naturally occurring in the human population with wide tissue tropism, non-integrating, non-pathogenic, low immunogenicity, infectivity of post-mitotic cells and relative ease of production, when compared to other viral systems, have ushered in the rapid expansion for human use. Gene delivery vectors based on adeno-associated virus (AAV), including rAAV, have re-emerged as safe and effective for broad applications.

The use of AAV (e.g., rAAV) in the clinical setting has underscored the urgent need for production and purification systems capable of generating very large amounts of pure AAV particles that also have sufficient viral titers suitable for use in gene therapy. Current techniques, however, are not able to remove all impurities, such as residual levels of proteins and nucleic acids that derive from the components of the production system within which the vector product is generated. Thus, there is an unmet need for the production of highly pure AAV particles, as well as compositions of AAV particles with high viral titers.

### SUMMARY

In one aspect, provided herein is a method for purifying adeno-associated viral (AAV) particles, said method comprising: (a) contacting a supernatant comprising AAV particles with a composition comprising a chromatin-DNA nuclease; and (b) purifying the AAV particles.

In one embodiment, purifying comprises centrifugation, chromatography, filtration, or a combination thereof. In one embodiment, centrifugation comprises density gradient centrifugation, ultracentrifugation, or a combination thereof. In one embodiment, chromatography comprises affinity chromatography, ion exchange chromatography, size exclusion chromatography, hydrophobic interaction chromatography, or a combination thereof.

In one embodiment, the method further comprises incubating the supernatant comprising AAV particles with a solid support for a sufficient amount of time to bind the AAV particles.

In one embodiment, the method further comprises washing the solid support. In one embodiment, the washing comprises a high pH buffer. In one embodiment, the high pH buffer is 15 greater than pH 9.0. In one embodiment, the high pH buffer is between pH 9.0 and pH 11. In one embodiment, the high pH buffer is about pH 9.5. In one embodiment, the high pH buffer is about pH 10.2. In one embodiment, the high pH buffer is about pH 10.3. In one embodiment, the high pH buffer is about pH 10.4.

In one embodiment, the method comprises one or more affinity chromatography purifications. In one embodiment, the affinity chromatography comprises ion exchange chromatography. In one embodiment, the ion exchange chromatography comprises anion exchange chromatography.

In one embodiment, the supernatant is a clarified supernatant.

In one embodiment, the composition of step (a) further comprises Benzonase®.

In one embodiment, the incubation is for about 10 minutes to about 1 hour. In one embodiment, the incubation is for about 20 minutes to about 40 minutes. In one embodiment, the incubation is for about 30 minutes.

In one embodiment, the chromatin-DNA nuclease is micrococcal nuclease (MNase). In one embodiment, the concentration of the MNase in the supernatant is greater than 2.5 units/mL. In one embodiment, the concentration of the MNase in the supernatant is greater than 10 units/mL. In one embodiment, the concentration of the MNase in the supernatant is about 30 units/mL to about 100 units/mL. In one embodiment, the concentration of the MNase in the supernatant is about 60 units/mL. In one embodiment, the MNase is incubated with the solid support containing bound AAV particles.

In one embodiment, the AAV particles are eluted from the solid support using a low pH buffer. In one embodiment, the elution further comprises a high pH buffer prior to the low pH buffer. In one embodiment, the low pH buffer is less than about pH 3.0. In one embodiment, the low pH buffer is about pH 1.5 to about pH 2.5. In one embodiment, the low pH buffer is about pH 1.5. In one embodiment, the low pH buffer is about pH 2.5.

In one embodiment, the low pH buffer is a citrate buffer, glycine buffer, or a phosphoric acid buffer. In one embodiment, the low pH buffer is a citrate buffer. In one embodiment, the low pH buffer is a phosphoric acid buffer.

In one embodiment, the affinity chromatography purification comprises two affinity chromatography purifications. In one embodiment, the method comprises an affinity chromatography purification followed by an anion-exchange chromatography.

In one embodiment, the elution further comprises neutralizing the pH of the low pH buffer. In one embodiment, neutralizing comprises adding Bis-Tris-Propane (BTP) or a Tris buffer.

In one embodiment, the elution further comprises ethanol. In one embodiment, the ethanol is about 5% to about 40%. In one embodiment, the ethanol is about 10% to about 30%. In one embodiment, the ethanol is about 15% to about 25%. In one embodiment, the ethanol is about 20% ethanol.

In one embodiment, the purified AAV particles are substantially free of chromatin-associated DNA, when compared to non-MNase contacted purified AAV particles.

In one embodiment, the purified AAV particles are substantially free of host-cell DNA, when compared to non-MNase contacted purified AAV particles. In one embodiment, host-cell DNA concentration is less than 2 ng/mL. In one embodiment, the host-cell DNA concentration is less than 1.5 ng/mL. In one embodiment, the host-cell DNA concentration is less than 1 ng/mL.

In one embodiment, the purified AAV particles are substantially free of host cell proteins, when compared to non-MNase contacted purified AAV particles. In one embodiment, purified AAV particles are substantially free of a DNA binding protein, when compared to non-MNase contacted purified AAV particles. In one embodiment, the DNA binding protein comprises a histone.

In one embodiment, the purified AAV particles are substantially free of macroscopic and microscopic impurities.

In one embodiment, the purified AAV particles have an increased viral titer, when compared to non-MNase contacted purified AAV particles. In one embodiment, the viral titer comprises a physical titer. In one embodiment, the viral titer comprises a functional titer. In one embodiment, the viral titer is increased about 2 fold to about 100 fold. In one embodiment, the viral titer is increased about 2 fold or greater. In one embodiment, the viral titer is increased about 3 fold or greater. In one embodiment, the viral titer is increased about 7 fold or greater. In one embodiment, the viral titer is increased about 80 fold or greater.

In one embodiment, the purified AAV particles comprise an increased viral titer ratio of a product AAV particle fraction to a post-product AAV particle fraction, when compared to non-MNase contacted purified AAV particles. In one embodiment, the viral titer ratio is increased about 2 fold or greater. In one embodiment, the viral titer ratio is increased about 5 fold or greater. In one embodiment, the viral titer ratio is increased about 10 fold or greater. In one embodiment, the viral titer ratio is increased about 25 fold or greater.

In one embodiment, the purified AAV particles have a melting temperature (Tm) within less than about 10° C. of an aggregation temperature (Tagg), as measured by dynamic light scatter (DLS). In one embodiment, the purified AAV particles have a Tm within less than about 5° C. of an aggregation temperature (Tagg), as measured by dynamic light scatter (DLS). In one embodiment, the purified AAV particles have a melting temperature (Tm) within less than 2° C. of an aggregation temperature (Tagg), as measured by dynamic light scatter (DLS).

In one embodiment, the purified AAV particles comprise a full-to-empty capsid ratio of greater than about 50%. In one embodiment, the purified AAV particles comprise a full-to-empty capsid ratio of greater than about 60%. In one embodiment, the purified AAV particles comprise a full-to-empty capsid ratio of greater than about 70%.

In one embodiment, the purified AAV particles comprise a AAV particle post-product fraction with a reduced absorbance at 260 nm, when compared to non-MNase contacted purified AAV particles. In one embodiment, the purified AAV particles comprise a AAV particle post-product fraction with a reduced absorbance at 280 nm, when compared to non-MNase contacted purified AAV particles.

In one aspect, provided herein is a method for increasing a viral titer of AAV particles, said method comprising (a) contacting a supernatant comprising AAV particles with a composition comprising a chromatin-DNA nuclease; and (b) purifying the AAV particles.

In one embodiment, the viral titer comprises a physical viral titer, a functional viral titer, or both. In one embodiment, the viral titer comprises a physical viral titer. In one embodiment, the viral titer comprises a functional viral titer.

In one embodiment, purifying comprises centrifugation, chromatography, filtration, or a combination thereof. In one embodiment, the centrifugation comprises density gradient centrifugation, ultracentrifugation, or a combination thereof. In one embodiment, the chromatography comprises affinity chromatography, ion exchange chromatography, size exclusion chromatography, hydrophobic interaction chromatography, or a combination thereof.

In one embodiment, the method further comprises incubating the supernatant comprising AAV particles with a solid support for a sufficient amount of time to bind the AAV particles.

In one embodiment, the method further comprises washing the solid support. In one embodiment, the washing comprises a high pH buffer. In one embodiment, the high pH buffer is greater than pH 9.0. In one embodiment, the high pH buffer is between pH 9.0 and pH 11. In one embodiment, the high pH buffer is about pH 9.5. In one embodiment, the high pH buffer is about pH 10.2. In one embodiment, the high pH buffer is about pH 10.3. In one embodiment, the high pH buffer is about pH 10.4.

In one embodiment, the method comprises one or more affinity chromatography purifications. In one embodiment, the affinity chromatography comprises ion exchange chromatography. In one embodiment, the ion exchange chromatography comprises anion exchange chromatography.

In one embodiment, the supernatant is a clarified supernatant.

In one embodiment, the composition of step (a) further comprises Benzonase®.

In one embodiment, the incubation is for about 10 minutes to about 1 hour. In one embodiment, the incubation is for about 20 minutes to about 40 minutes. In one embodiment, the incubation is for about 30 minutes.

In one embodiment, the chromatin-DNA nuclease is micrococcal nuclease (MNase). In one embodiment, the concentration of the MNase in the supernatant is greater than 2.5 units/mL. In one embodiment, the concentration of the MNase in the supernatant is greater than 10 units/mL. In one embodiment, the concentration of the MNase in the supernatant is about 30 units/mL to about 100 units/mL. In one embodiment, the concentration of the MNase in the supernatant is about 60 units/mL. In one embodiment, the MNase is incubated with the solid support containing bound AAV particles.

In one embodiment, the AAV particles are eluted from the solid support using a low pH buffer. In one embodiment, the elution further comprises a high pH buffer prior to the low pH buffer. In one embodiment, the low pH buffer is less than about pH 3.0. In one embodiment, the low pH buffer is about pH 1.5 to about pH 2.5. In one embodiment, the low pH buffer is about pH 1.5. In one embodiment, the low pH buffer is about pH 2.5.

In one embodiment, the low pH buffer is a citrate buffer, glycine buffer, or a phosphoric acid buffer. In one embodiment, the low pH buffer is a citrate buffer. In one embodiment, the low pH buffer is a phosphoric acid buffer.

In one embodiment, the affinity chromatography purification comprises two affinity chromatography purifications. In one embodiment, the method comprises an affinity chromatography purification followed by an anion-exchange chromatography.

In one embodiment, the elution further comprises neutralizing the pH of the low pH buffer. In one embodiment, neutralizing comprises adding Bis-Tris-Propane (BTP) or a Tris buffer.

In one embodiment, the elution further comprises ethanol. In one embodiment, the ethanol is about 5% to about 40%. In one embodiment, the ethanol is about 10% to about 30%. In one embodiment, the ethanol is about 15% to about 25%. In one embodiment, the ethanol is about 20% ethanol.

In one embodiment, the purified AAV particles are substantially free of chromatin-associated DNA, when compared to non-MNase contacted purified AAV particles.

In one embodiment, the purified AAV particles are substantially free of host-cell DNA, when compared to non-MNase contacted purified AAV particles. In one embodiment, the host-cell DNA concentration is less than 2 ng/mL. In one embodiment, the host-cell DNA concentration is less than 1.5 ng/mL. In one embodiment, the host-cell DNA concentration is less than 1 ng/mL.

In one embodiment, the purified AAV particles are substantially free of host cell proteins, when compared to non-MNase contacted purified AAV particles. In one embodiment, purified AAV particles are substantially free of a DNA binding protein, when compared to non-MNase contacted purified AAV particles. In one embodiment, the DNA binding protein comprises a histone.

In one embodiment, the purified AAV particles are substantially free of macroscopic and microscopic impurities.

In one embodiment, the viral titer is increased about 2 fold to about 100 fold. In one embodiment, the viral titer is increased about 2 fold or greater. In one embodiment, the viral titer is increased about 3 fold or greater. In one embodiment, the viral titer is increased about 7 fold or greater. In one embodiment, the viral titer is increased about 80 fold or greater.

In one embodiment, the purified AAV particles comprise an increased viral titer ratio of a product AAV particle fraction to a post-product AAV particle fraction, when compared to non-MNase contacted purified AAV particles. In one embodiment, the viral titer ratio is increased about 2 fold or greater. In one embodiment, the viral titer ratio is increased about 5 fold or greater. In one embodiment, the viral titer ratio is increased about 10 fold or greater. In one embodiment, the viral titer ratio is increased about 25 fold or greater.

In one embodiment, the purified AAV particles have a melting temperature (Tm) within less than about 10° C. of an aggregation temperature (Tagg), as measured by dynamic light scatter (DLS). In one embodiment, the purified AAV particles have a Tm within less than about 5° C. of an aggregation temperature (Tagg), as measured by dynamic light scatter (DLS). In one embodiment, the purified AAV particles have a melting temperature (Tm) within less than 2° C. of an aggregation temperature (Tagg), as measured by dynamic light scatter (DLS).

In one embodiment, the purified AAV particles comprise a full-to-empty capsid ratio of greater than about 50%. In one embodiment, the purified AAV particles comprise a full-to-empty capsid ratio of greater than about 60%. In one embodiment, the purified AAV particles comprise a full-to-empty capsid ratio of greater than about 70%.

In one embodiment, the purified AAV particles comprise a AAV particle post-product fraction with a reduced absorbance at 260 nm, when compared to non-MNase contacted purified AAV particles. In one embodiment, the purified AAV particles comprise a AAV particle post-product fraction with a reduced absorbance at 280 nm, when compared to non-MNase contacted purified AAV particles.

In one aspect, provided herein is a composition of purified AAV particles, wherein the AAV particles have been purified by a purification method comprising a chromatin-DNA nuclease. In one embodiment, the purification method comprises centrifugation, chromatography, filtration, or a combination thereof. In one embodiment, centrifugation comprises density gradient centrifugation, ultracentrifugation, or a combination thereof. In one embodiment, the chromatography comprises affinity chromatography, ion exchange chromatography, size exclusion chromatography, hydrophobic interaction chromatography, or a combination thereof.

In one embodiment, the purification method further comprises incubating a supernatant comprising AAV particles with a solid support for a sufficient amount of time to bind the AAV particles.

In one embodiment, the purification method further comprises washing the solid support. In one embodiment, the washing comprises a high pH buffer. In one embodiment, the high pH buffer is greater than pH 9.0. In one embodiment, the high pH buffer is between pH 9.0 and pH 11. In one embodiment, the high pH buffer is about pH 9.5. In one embodiment, the high pH buffer is about pH 10.2. In one embodiment, the high pH buffer is about pH 10.3. In one embodiment, the high pH buffer is about pH 10.4

In one embodiment, the purification method comprises one or more affinity chromatography purifications. In one embodiment, the affinity chromatography comprises ion exchange chromatography. In one embodiment, the ion exchange chromatography comprises anion exchange chromatography.

In one embodiment, the supernatant is a clarified supernatant.

In one embodiment, the purification method further comprises Benzonase®.

In one embodiment, the purification method comprises elution with a low pH buffer. In one embodiment, the purification method further comprises a high pH buffer prior to the low pH buffer. In one embodiment, the low pH buffer is less than about pH 3.0. In one embodiment, the low pH buffer is about pH 1.5 to about pH 2.5. In one embodiment, the low pH buffer is about pH 1.5. In one embodiment, the low pH buffer is about pH 2.5.

In one embodiment, the low pH buffer is a citrate buffer, glycine buffer, or a phosphoric acid buffer. In one embodiment, the low pH buffer is a citrate buffer. In one embodiment, the low pH buffer is a phosphoric acid buffer.

In one embodiment, the purification method comprises two affinity chromatography purifications. In one embodiment, the purification method comprises an affinity chromatography purification followed by an anion-exchange chromatography.

In one embodiment, the elution further comprises neutralizing the pH of the low pH buffer. In one embodiment, neutralizing comprises adding Bis-Tris-Propane (BTP) or a Tris buffer.

In one embodiment, the elution further comprises ethanol. In one embodiment, the ethanol is about 5% to about 40%. In one embodiment, the ethanol is about 10% to about 30%. In one embodiment, the ethanol is about 15% to about 25%. In one embodiment, the ethanol is about 20% ethanol.

In one embodiment, the composition is substantially free of an impurity, when compared to a composition purified by a method not comprising a chromatin-DNA nuclease.

In one embodiment, the composition is substantially free of chromatin-associated DNA, when compared to a composition purified by a method not comprising a chromatin-DNA nuclease.

In one embodiment, the composition is substantially free of host-cell DNA, when compared to a composition purified by a method not comprising a chromatin-DNA nuclease. In one embodiment, the host-cell DNA concentration is less than 2 ng/mL. In one embodiment, the host-cell DNA concentration is less than 1.5 ng/mL. In one embodiment, the host-cell DNA concentration is less than 1 ng/mL.

In one embodiment, the composition is substantially free of host cell proteins, when compared to a composition not contacted with a chromatin-DNA nuclease. In one embodiment, the composition is substantially free of a DNA binding protein, when compared to a composition not contacted with a chromatin-DNA nuclease. In one embodiment, the DNA binding protein comprises a histone.

In one embodiment, the composition is substantially free of macroscopic and microscopic impurities.

In one embodiment, the composition comprises a reduced post-product AAV particle fraction peak as measured by anion exchange chromatogram, when compared to a composition not contacted with a chromatin-DNA nuclease.

In one embodiment, the composition comprises an increased viral titer, when compared to a composition not contacted with a chromatin-DNA nuclease. In one embodiment, the viral titer comprises a physical titer. In one embodiment, the viral titer comprises a functional titer. In one embodiment, the viral titer is increased about 2 fold to about 100 fold. In one embodiment, the viral titer is increased about 2 fold or greater. In one embodiment, the viral titer is increased about 3 fold or greater. In one embodiment, the viral titer is increased about 7 fold or greater. In one embodiment, the viral titer is increased about 80 fold or greater.

In one embodiment, the composition comprises an increased viral titer ratio of a product AAV particle fraction to a post-product AAV particle fraction, when compared to a composition not contacted with a chromatin-DNA nuclease. In one embodiment, the viral titer ratio is increased about 2 fold or greater. In one embodiment, the viral titer ratio is increased about 5 fold or greater. In one embodiment, the viral titer ratio is increased about 10 fold or greater. In one embodiment, the viral titer ratio is increased about 25 fold or greater.

In one embodiment, the purified AAV particles have a melting temperature (Tm) within less than about 10° C. of an aggregation temperature (Tagg), as measured by dynamic light scatter (DLS). In one embodiment, the purified AAV particles have a Tm within less than about 5° C. of an aggregation temperature (Tagg), as measured by dynamic light scatter (DLS). In one embodiment, the purified AAV particles have a melting temperature (Tm) within less than 2° C. of an aggregation temperature (Tagg), as measured by dynamic light scatter (DLS).

In one embodiment, the composition comprises a full-to-empty capsid ratio of greater than about 50%. In one embodiment, the composition comprises a full-to-empty capsid ratio of greater than about 60%. In one embodiment, the composition comprises a full-to-empty capsid ratio of greater than about 70%.

In one embodiment, the purified AAV particles comprise a AAV particle post-product fraction with a reduced absorbance at 260 nm, when compared to a composition not contacted with a chromatin-DNA nuclease. In one embodiment, the purified AAV particles comprise a AAV particle post-product fraction with a reduced absorbance at 280 nm, when compared to a composition not contacted with a chromatin-DNA nuclease.

In one embodiment, the chromatin-DNA nuclease is MNase.

In one aspect, provided herein is a composition for use in producing an AAV particle that is substantially free of chromatin-associated DNA, the composition comprising: (a) a supernatant comprising AAV particles; and (b) a chromatin-DNA nuclease.

In one embodiment, the composition further comprises Benzonase®.

In one embodiment, the chromatin-DNA nuclease is micrococcal nuclease (MNase). In one embodiment, the concentration of the MNase in the supernatant is greater than 2.5 units/mL. In one embodiment, the concentration of the MNase in the supernatant is greater than 10 units/mL. In one embodiment, the concentration of the MNase in the supernatant is about 30 units/mL to about 100 units/mL. In one embodiment, the concentration of the MNase in the supernatant is about 60 units/mL. In one embodiment, the MNase is present in a sufficient amount to digest chromatin associated with an AAV particle.

In another aspect, provided herein is a composition, comprising: (a) a supernatant comprising AAV particles; and (b) a chromatin-DNA nuclease.

In one embodiment, the composition further comprises Benzonase®.

In one embodiment, the chromatin-DNA nuclease is micrococcal nuclease (MNase). In one embodiment, the concentration of the MNase in the supernatant is greater than 2.5 units/mL. In one embodiment, the concentration of the MNase in the supernatant is greater than 10 units/mL. In one embodiment, the concentration of the MNase in the supernatant is about 30 units/mL to about 100 units/mL. In one embodiment, the concentration of the MNase in the supernatant is about 60 units/mL. In one embodiment, the MNase is present in a sufficient amount to digest chromatin associated with an AAV particle.

In one embodiment, the MNase is present in a sufficient amount to reduce AAV particle impurities. In one embodiment, the AAV particle impurities comprise one or more of a host-cell DNA, a host-cell protein, a chromatin-associated DNA, and a DNA binding protein. In one embodiment, the AAV particle impurities comprise macroscopic and microscopic impurities. In one embodiment, the DNA binding protein comprises a histone.

In one embodiment, the MNase is present in an amount sufficient to increase a viral titer of AAV particles. In one embodiment, the viral titer comprises a physical viral titer, a functional viral titer, or both. In one embodiment, the viral titer comprises a physical titer. In one embodiment, the viral titer comprises a functional titer. In one embodiment, the viral titer is increased about 2 fold to about 100 fold. In one embodiment, the viral titer is increased about 2 fold or greater. In one embodiment, the viral titer is increased about 3 fold or greater. In one embodiment, the viral titer is increased about 7 fold or greater. In one embodiment, the viral titer is increased about 80 fold or greater.

In one embodiment, the MNase is present in a sufficient amount to increase a viral titer ratio of a product AAV particle fraction to a post-product AAV particle fraction.

In one embodiment, the viral titer ratio is increased about 2 fold or greater. In one embodiment, the viral titer ratio is increased about 5 fold or greater. In one embodiment, the viral titer ratio is increased about 10 fold or greater. In one embodiment, the viral titer ratio is increased about 25 fold or greater.

In one embodiment, the MNase is present in an amount sufficient to increase a full-to-empty capsid ratio. In one embodiment, the full-to-empty capsid ratio is greater than about 50%. In one embodiment, the full-to-empty capsid ratio is greater than about 60%. In one embodiment, the full-to-empty capsid ratio is greater than about 70%.

In one embodiment, the MNase is present in an amount sufficient to decrease a AAV particle post-product fraction, as measured by absorbance at 260 nm. In one embodiment, the MNase is present in an amount sufficient to decrease a AAV particle post-product fraction, as measured by absorbance at 280 nm.

In one embodiment, the MNase is present in an amount sufficient to produce purified AAV particles comprising a melting temperature (Tm) within less than about 10° C. of an aggregation temperature (Tagg), as measured by dynamic light scatter (DLS).

In one embodiment, the MNase is present in an amount sufficient to produce purified AAV particles comprising a melting temperature (Tm) within less than about 5° C. of an aggregation temperature (Tagg), as measured by dynamic light scatter (DLS).

In one embodiment, the MNase is present in an amount sufficient to produce purified AAV particles comprising a melting temperature (Tm) within less than about 2° C. of an aggregation temperature (Tagg), as measured by dynamic light scatter (DLS).

In one aspect, provided herein is a kit comprising, (a) Benzonase®; and (b) a chromatin-DNA nuclease.

In one embodiment, the chromatin-DNA nuclease is micrococcal nuclease (MNase). In one embodiment, the concentration of the MNase in the supernatant is greater than 2.5 units/mL. In one embodiment, the concentration of the MNase in the supernatant is greater than 10 units/mL. In one embodiment, the concentration of the MNase in the supernatant is about 30 units/mL to about 100 units/mL. In one embodiment, the concentration of the MNase in the supernatant is about 60 units/mL. In one embodiment, the MNase is present in a sufficient amount to digest chromatin associated with an AAV particle.

In one embodiment, the MNase is present in a sufficient amount to reduce AAV particle impurities. In one embodiment, the AAV particle impurities comprise one or more of a host-cell DNA, a host-cell protein, a chromatin-associated DNA, and a DNA binding protein. In one embodiment, the AAV particle impurities comprise macroscopic and microscopic impurities. In one embodiment, the DNA binding protein comprises a histone.

In one embodiment, the MNase is present in an amount sufficient to increase a viral titer of AAV particles. In one embodiment, the viral titer comprises a physical viral titer, a functional viral titer, or both. In one embodiment, the viral titer comprises a physical titer. In one embodiment, the viral titer comprises a functional titer. In one embodiment, the viral titer is increased about 2 fold to about 100 fold. In one embodiment, the viral titer is increased about 2 fold or greater. In one embodiment, the viral titer is increased about 3 fold or greater. In one embodiment, the viral titer is increased about 7 fold or greater. In one embodiment, the viral titer is increased about 80 fold or greater.

In one embodiment, the MNase is present in a sufficient amount to increase a viral titer ratio of a product AAV particle fraction to a post-product AAV particle fraction.

In one embodiment, the viral titer ratio is increased about 2 fold or greater. In one embodiment, the viral titer ratio is increased about 5 fold or greater. In one embodiment, the viral titer ratio is increased about 10 fold or greater. In one embodiment, the viral titer ratio is increased about 25 fold or greater.

In one embodiment, the MNase is present in an amount sufficient to increase a full-to-empty capsid ratio. In one embodiment, the full-to-empty capsid ratio is greater than about 50%. In one embodiment, the full-to-empty capsid ratio is greater than about 60%. In one embodiment, the full-to-empty capsid ratio is greater than about 70%.

In one embodiment, the MNase is present in an amount sufficient to decrease a AAV particle post-product fraction, as measured by absorbance at 260 nm. In one embodiment, the MNase is present in an amount sufficient to decrease a AAV particle post-product fraction, as measured by absorbance at 280 nm.

In one embodiment, the MNase is present in an amount sufficient to produce purified AAV particles comprising a melting temperature (Tm) within less than about 10° C. of an aggregation temperature (Tagg), as measured by dynamic light scatter (DLS).

In one embodiment, the MNase is present in an amount sufficient to produce purified AAV particles comprising a melting temperature (Tm) within less than about 5° C. of an aggregation temperature (Tagg), as measured by dynamic light scatter (DLS).

In one embodiment, the MNase is present in an amount sufficient to produce purified AAV particles comprising a melting temperature (Tm) within less than about 2° C. of an aggregation temperature (Tagg), as measured by dynamic light scatter (DLS).

In one aspect, provided herein is a composition comprising a means for decreasing an impurity in purified AAV particles.

In one embodiment, the impurity is selected from the group consisting of a host-cell DNA, a host-cell protein, a chromatin-associated DNA, and a DNA binding protein. In one embodiment, the DNA binding protein comprises a histone. In one embodiment, the impurity is a macroscopic impurity, a microscopic impurity, or both.

In another aspect, provided herein is a composition comprising a means for increasing a viral titer of AAV particles. In one embodiment, the viral titer comprises a physical viral titer, a functional viral titer, or both. In one embodiment, the viral titer comprises a physical viral titer. In one embodiment, the viral titer comprises a functional viral titer. In one embodiment, the viral titer is increased about 2 fold to about 100 fold. In one embodiment, the viral titer is increased about 2 fold or greater. In one embodiment, the viral titer is increased about 3 fold or greater. In one embodiment, the viral titer is increased about 7 fold or greater. In one embodiment, the viral titer is increased about 80 fold or greater.

In yet another aspect, provided herein is a composition comprising a means for increasing a viral titer ratio of a product AAV particle fraction to a post-product AAV particle fraction. In one embodiment, the viral titer ratio is increased about 2 fold or greater. In one embodiment, the viral titer ratio is increased about 5 fold or greater. In one embodiment, the viral titer ratio is increased about 10 fold or greater. In one embodiment, the viral titer ratio is increased about 25 fold or greater.

In another aspect, provided herein is a composition comprising a means for increasing the full-to-empty capsid ratio of AAV particles. In one embodiment, the full-to-empty capsid ratio is greater than about 50%. In one embodiment, the full-to-empty capsid ratio is greater than about 60%. In one embodiment, the full-to-empty capsid ratio is greater than about 70%.

In one aspect, provided herein is a composition comprising a means to decrease a AAV particle post-product fraction, as measured by absorbance at 260 nm.

In one aspect, provided herein is a composition comprising a means to decrease a AAV particle post-product fraction, as measured by absorbance at 280 nm.

In one aspect, provided herein is a composition comprising a first means to remove a DNA binding protein extra-virally complexed to an AAV particle and a second means to remove residual host production cell nucleic acids and/or proteins.

In one aspect, provided herein is a method of purifying an AAV particle comprising (i) a step for removing a DNA binding protein extra-virally complexed to an AAV particle. In one embodiment, the method further comprises (ii) a second step for removing residual host production cell nucleic acids and/or proteins. In one embodiment, the method further comprises (iii) a third step for increasing a viral titer.

In one aspect, provided herein is a system comprising a means for making and obtaining a purified AAV particle substantially free of an impurity. In one embodiment, the impurity is selected from the group consisting of a host-cell DNA, a host-cell protein, a chromatin-associated DNA, and a DNA binding protein. In one embodiment, the DNA binding protein comprises a histone. In one embodiment, the impurity is a macroscopic impurity, a microscopic impurity, or both.

In one aspect, provided herein is a system comprising a means for making and obtaining AAV particles with an increased viral titer.

In one embodiment, the viral titer comprises a physical viral titer, a functional viral titer, or both. In one embodiment, the viral titer comprises a physical titer. In one embodiment, the viral titer comprises a functional titer. In one embodiment, the viral titer is increased about 2 fold to about 100 fold. In one embodiment, the viral titer is increased about 2 fold or greater. In one embodiment, the viral titer is increased about 3 fold or greater. In one embodiment, the viral titer is increased about 7 fold or greater. In one embodiment, the viral titer is increased about 80 fold or greater.

In one aspect, provided herein is a system comprising a means for increasing a viral titer ratio of a product AAV particle fraction to a post-product AAV particle fraction. In one embodiment, the viral titer ratio is increased about 2 fold or greater. In one embodiment, the viral titer ratio is increased about 5 fold or greater. In one embodiment, the viral titer ratio is increased about 10 fold or greater. In one embodiment, the viral titer ratio is increased about 25 fold or greater.

In one aspect, provided herein is a system comprising a means for increasing the full-to-empty capsid ratio of AAV particles. In one embodiment, the full-to-empty capsid ratio is greater than about 50%. In one embodiment, the full-to-empty capsid ratio is greater than about 60%. In one embodiment, the full-to-empty capsid ratio is greater than about 70%.

In one aspect, provided herein is a system comprising a means to decrease a AAV particle post-product fraction, as measured by absorbance at 260 nm.

In one aspect, provided herein is a system comprising a means to decrease a AAV particle post-product fraction, as measured by absorbance at 280 nm.

In one aspect, provided herein is a system comprising a first means to remove DNA binding proteins extra-virally complexed to AAV particles and a second means to remove residual nucleic acids from a host production.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A and FIG. 1B illustrate that MNase treatment does not affect affinity chromatography of AAV particles. Supernatant containing viral particles was bound to the column at normal pH (7.5), and Benzonase® was added with MNase (FIG. 1A) or without MNase (FIG. 1B) directly to the AAVX-containing bound virus. Addition of MNase in this step did not affect the shape of the chromatogram or yield of viral particles.

FIG. 2A and FIG. 2B illustrate that the addition of MNase released DNA binding proteins extra-virally complexed to AAV particles. FIG. 2A shows gel electrophoresis and silver-staining (right) after collecting the sample fraction corresponding with the product peak (left) using affinity chromatography after 30 minutes of no treatment (first lane), 2.5 U/mL of MNase, or 60 U/mL of MNase. 60 U/mL of MNase yielded a strong band around ˜10 kDa, which is around the predicted molecular weight of a histone. Samples taken of post-product peak fractions and electrophoresis was performed to visualize any chromatin that may be present in the sample (FIG. 6B). The results indicated treatment with MNase during affinity capture chromatography is sufficient to remove chromatin from the rAAV8 particles. Lane 1 is a 1 kb ladder, Lane 2 is rAAV8 particles produced in suspension Expi293F™ cells using the ExpiFectamine™ 293 Transfection Kit, Lane 3 is a 1:10 dilution of the sample in Lane 2, Lane 4 is rAAV8 particles produced in suspension Expi293F™ cells without the transfection kit, Lane 5 is a 1:10 dilution of the sample in Lane 4, Lane 6 is rAAV8 particles produced in suspension Expi293F™ cells without the transfection kit and digested with 60 U/mL MNase at 25° C. for 30 minutes, and Lane 7 is a 1:10 dilution of the sample in Lane 6.

FIG. 3 illustrates that minimal cell death during AAV particle production in adherent cell culture produces an insignificant post-product peak during affinity capture.

FIG. 4A and FIG. 4B illustrate that 60 U/mL of MNase treatment (FIG. 4B) for chromatin digestion enhances AAV particle purification, as compared to non-MNase treaded AAV particle purification (FIG. 4A). The large 260 nm (RNA/DNA) absorbance contribution to the post-product peak is greatly reduced, as is the 280 nm (protein) absorbance peak, after MNase treatment.

FIG. 5A and FIG. 5B illustrate overlays of the chromatogram from rAAV8 particles containing samples treated with or without MNase. Addition of MNase caused a significant reduction in post-product peak heights for DNA/RNA (260 nm) (FIG. 5A) and protein (280 nm) (FIG. 5B), which indicated that post-product peaks are AAV particles containing extra-virally associated chromatin and that MNase treatment enhanced AAV particle purification.

FIG. 6 illustrates that MNase treatment allows chromatin to be digested and removed during normal AAV particle purification operations. Silver stain analysis of post-product peaks after anion exchange chromatography revealed protein impurities present in the non-MNase digested samples, including a band at ˜20 kDa (FIG. 6A). Lane 1 is rAAV8 particles produced in suspension Expi293F™ cells using the ExpiFectamine™ 293 Transfection Kit Enhancer, Lane 2 is rAAV8 particles produced in suspension Expi293F™ cells without Enhancer, and Lane 3 is rAAV8 particles produced in suspension Expi293F™ cells without Enhancer and digested with 60 U/mL MNase at 25° C. for 30 minutes.

FIG. 7A-FIG. 7C illustrate that increasing the amount of MNase from no MNase (FIG. 7A) or 2.5 U/mL of MNase (FIG. 7B) to 60 U/mL of MNase (FIG. 7C) reduced chromatin-associated AAV particles in anion exchange chromatography polish step.

FIG. 8 illustrates that examination of the post-product peaks demonstrated visible precipitate in the non-MNase treated samples produced either using the ExpiFectamine™ 293 Transfection Kit Enhancer or without the enhancer. However, MNase digestion prevented aggregation and precipitation of viral particles.

FIG. 9A-FIG. 9F illustrate that MNase treatment increased amounts of DNA in the product peak fractions, as compared to non-MNase treated samples using three different types of elution. High/Low pH elution without MNase (FIG. 9A), High/Low pH elution with MNase (FIG. 9D), citrate elution without MNase (FIG. 9B), citrate elution with MNase (FIG. 9E), low pH elution without MNase (FIG. 9C), and low pH elution with MNase (FIG. 9F). Lane 1 (enzyme load, “L”), lane 2 (enzyme washout, “W”), lane 3 (high pH wash “H” or empty lane “X”), lane 4 (anion exchange product peak “P”), lane 5 (anion exchange post-product peak “PP”), and lane 6 (AAVX strip peak “S”).

FIG. 10A-FIG. 10C illustrate that MNase treatment increased viral titers (FIG. 10A), genome copies/cell (FIG. 10B), and total genome copies (FIG. 10C), and reduced the amount of post-product produced, as compared to non-MNase treated samples and samples generated using the ExpiFectamine™ 293 Transfection Kit Enhancer.

FIG. 11A and FIG. 11B illustrate that an increase in genome copies per cell (GC/cell) (FIG. 11A), and total genome copies (FIG. 11B) was consistently observed in MNase treated samples (circle), relative to no MNase treated samples (square), for each of the three different elution buffers: citrate, low pH, and low/high pH.

FIG. 12 illustrates that MNase treatment increased the infectivity of the high/low pH elution product fractions (top right), relative to no MNase treated product fractions (top left), and decreased the infectivity of the MNase treated post-product fraction (bottom right), relative to no MNase treated post-product fractions (bottom left)

FIG. 13 illustrates that MNase treatment increased the infectivity of the citrate elution product fractions (top right), relative to no MNase treated product fractions (top left), and decreased the infectivity of the MNase treated post-product fraction (bottom right), relative to no MNase treated post-product fractions (bottom left).

FIG. 14 illustrates that MNase treatment increased the infectivity of the low pH elution product fractions (top right), relative to no MNase treated product fractions (top left), and decreased the infectivity of the MNase treated post-product fraction (bottom right), relative to no MNase treated post-product fractions (bottom left).

FIG. 15A and FIG. 15B illustrate that non-enzymatic or Benzonase® only treated samples contain impurities, as measured by silver staining. FIG. 15A depicts purified AAV samples without on-column enzyme treatment, and FIG. 15B depicts samples with Benzonase® only treatment. PE=pre-lution peak; E=elution; R=retentate (100 kDa amicon); F=filtrate; S=strip. Marker sizes are in kDa.

FIG. 16A and FIG. 16B illustrate that a high pH wash buffer applied during affinity chromatography prior to Benzonase® and MNase treatment decreases the amount of impurities after AAV particle purification, as measured by silver staining. FIG. 16A depicts AAV samples subjected to high pH (9.5) wash+on-column Benzonase®/MNase treatment and citrate (pH 1.5) elution. FIG. 16B depicts AAV samples subjected to high pH (10.2) wash+on-column Benzonase®/MNase treatment and phosphoric acid (pH 1.5) elution. The three intense bands correspond with VP1, VP2, and VP3. W=wash peak; E=elution peak; R=retentate (100 kDa amicon); F=filtrate; S=strip peak. Marker sizes are in kDa.

FIG. 17A and FIG. 17B illustrate a summary of viral purification using the different purification conditions described in Table 5. FIG. 17A depicts a silver stain of the purified AAV particles. The three intense bands correspond with VP1, VP2, and VP3. FIG. 17B depicts a DNA agarose gel electrophoresis and the presence of the ITR-transgene contained within the AAV. C=citrate, pH 2.5; P=phosphoric acid, pH 1.5; “+¹”=pH 10.3; “+²”=pH 9.5; “+³”=pH 10.2; “+⁴”=pH 10.4; * ═(C₂H₅)NCL anion exchange elution.

FIG. 18 illustrates a scheme for how impurities and DNA can be removing to improve the purity of AAV particle purification.

FIG. 19A-FIG. 19C illustrate that modification of elution conditions to include ethanol can improve the recovery of virus from anion-exchange columns. FIG. 19A depicts samples eluted using phosphoric acid (pH 1.5) after Benzonase®, and a high pH wash. FIG. 19B depicts samples eluted using phosphoric acid (pH 1.5) after Benzonase® plus MNase, and a high pH wash. FIG. 19C illustrates samples eluted using phosphoric acid (pH 1.5) and 20% ethanol after Benzonase® plus MNase, and a high pH wash. Arrows indicate the residual virus detected after column stripping.

FIG. 20A-FIG. 20E illustrate results from dynamic light scatter (DLS) and protein aggregation (Tagg) and melting (Tm) curves following no enzyme treatment (FIG. 20A); Benzonase® only (FIG. 20B); Benzonase® and MNase (FIG. 20C); Benzonase®, no MNase, high pH wash, and phosphoric acid elution (FIG. 20D); and Benzonase®, MNase, high pH wash, and phosphoric acid elution (FIG. 20E).

FIG. 21A and FIG. 21B illustrate the raw counts (FIG. 21A) and concentration results (FIG. 21B) using AlphaLISA to detect host-cell DNA following purification without Benzonase® or MNase (“no enzyme”); (2) purification with Benzonase® and citrate elution (“B, citrate (affinity)”); (3) purification with Benzonase® and citrate elution (“B, citrate”); (4) purification with Benzonase®, MNase, and phosphoric acid elution (“B, M, Phos”); (5) purification with Benzonase®, high pH (pH 10.3) wash, and phosphoric acid elution (“B, pH 10.3, Phos”); and (6) purification with Benzonase®, MNase, and phosphoric acid elution (“B, M, pH 10.3, Phos”).

FIG. 22 illustrates gel electrophoresis and silver-staining after collecting the AAV particle sample fraction corresponding with the product peak following Protocol #1 (lane 1), the AAV particle sample fraction corresponding with the product peak following Protocol #2 (lane 2), and the AAV particle sample fraction corresponding with the post-product peak following Protocol #2 (lane 3).

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “purifying” is intended to mean any technique that is able to remove impurities (e.g., host cell proteins, chromatin, and/or nucleic acids) and enrich for AAV particles. Exemplary techniques for purifying an AAV particle include but are not limited to, for example, centrifugation (e.g., density gradient centrifugation, ultracentrifugation, or a combination thereof), chromatography (e.g., affinity chromatography, ion exchange chromatography, size exclusion chromatography, or hydrophobic interaction chromatography), filtration or a combination of such techniques. It is understood that purifying can include one-step purifying techniques, or multi-step purifying techniques that combine two or more types of purification techniques.

As used herein, the term “adeno-associated virus (AAV)” is intended to mean both naturally occurring, including all the different AAV serotypes, as well as non-naturally occurring forms of AAV (e.g., recombinant rAAV, and pseudotypes), and variants thereof. AAV viruses consist of the Rep gene (translated as Rep78, Rep68, Rep52, Rep40—required for the AAV life cycle), and the Cap gene (translated as VP1, VP2, VP3—capsid proteins).

As used herein, the term “viral particle” or “AAV particle,” is intended to mean the complete, infective form of the AAV virus outside a host cell, that contains nucleic acids and is surrounded by a protective coat of protein called a capsid.

As used herein, the term “titer” is intended to mean the quantity of virus in a given volume. A viral titer can include a “physical titer” or a “functional titer.” The physical titer is a measurement of how much virus is present, and is generally expressed as the number of viral particles per mL (VP/mL), or genome copies per mL (GC/mL). Functional titer, or infectious titer, is the measurement of how much virus actually infects a target cell and is generally expressed in the form of transduction units per mL (TU/mL), or for adenovirus as plaque-forming units per mL (pfu/mL) or infectious units per mL (ifu/mL). It is understood that functional titer will generally be lower than physical titer, usually by a factor of about 10 to about 100-fold.

As used herein, the term “sufficient amount” is intended to mean a quantity that is able to produce a desired effect or achieve a desired result, such as for example, binding of AAV particles to a solid support, such as an affinity support, or removing impurities (e.g., host cell proteins, chromatin, and/or nucleic acids) from a sample containing AAV particles.

As used herein, the term “substantially free” when used in reference to a sample of AAV particles is intended to mean that the sample of AAV particles includes less than about 50%, less than about 20%, less than about 10%, less than about 5% of an impurity, as compared to a matched negative control sample. Exemplary impurities include but are not limited to, host cell proteins, and extra-viral, chromatin-associated DNA. It is further understood that a sample can be “substantially free” of one or more impurities, but continue to have a small amount (e.g., undetectable level) of some impurities and that “substantially free” does not require complete removal of all impurities.

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, the types of purification techniques provided herein can be combined with any of the various exemplary methods for production of AAV particles provided herein. The application contemplates use of any of the applicable components in any combination, whether or not a particular combination is expressly described.

Provided herein are methods, compositions, kits, and systems for purifying adeno-associated viral (AAV) particles using a chromatin-DNA nuclease. Various purification techniques for purifying AAV particles for small-scale production (e.g., density gradient centrifugation) or large-scale production (e.g., affinity chromatography, including ion exchange chromatography; size exclusion chromatography; and hydrophobic interaction chromatography; or a combination of such techniques) are known in the art (see, e.g., Burova and Ioffe, Gene Therapy (2005) 12, S5-S17). However, even with techniques that generally yield high purity products, process-related impurities encountered in AAV particle manufacturing can be problematic. For example, as provided herein, the present disclosure provides that extra-viral, chromatin-associated AAV particles represent an important impurity that can be detected in the purified AAV particle products, which can cause visible precipitation of the purified products and be problematic for downstream applications of the AAV particles, such as, for example, limiting viral infectivity. Accordingly, the present disclosure that can be incorporated into any of the various methods known in the art for purifying AAV particles to enhance the purity and/or titer of the final purified product.

Chromatin is a complex of DNA, proteins, and associated proteins. The major proteins in chromatin are histones. Histones are a family of small, positively charged proteins termed H1, H2A, H2B, H3, and H4 that strongly adhere to negatively-charged DNA and form complexes called nucleosomes. The chromatin-associated with AAV particles is generally resistant to nucleases because the DNA is protected by histones and inaccessible to nucleases.

Chromatin-associated proteins include, for example, histone methyltransferases (e.g., the Polycomb group (PcG) protein EZH2; DOTL1; PRMT5), histone demethylases (e.g., LSD1, JmjC-Domain Containing Histone Demethylases), BET family of bromodomain-containing (e.g., BRD2, BRD3, and BRD4 and BRDT), among others. Additional chromatin modifying and DNA binding proteins include, for example, Zinc finger (ZnF) proteins or other DNA binding proteins.

Accordingly, the present disclosure provides that the addition of a specific type of nuclease, a chromatin-DNA nuclease (e.g., micrococcal nuclease; MNase), to any type of AAV particle purification method can significantly improve the quality of AAV particles recovered during down-stream purification, as well as the yield of AAV particles recovered.

Thus, in some embodiments, provided herein is a method for purifying AAV particles that includes (a) contacting the supernatant comprising AAV particles with a composition comprising a chromatin-DNA nuclease; and (b) purifying the AAV particles.

As described above, the AAV particles can be purified using various methods known in the art, and the chromatin-DNA nuclease can be combined with any type of AAV particle purification method. Generally, purification involves centrifugation, chromatography, or filtration, or possibly a combination thereof. In some embodiments, purification can include a two-step purification protocol, including, for example, two chromatographic steps or a combination of chromatography with ultracentrifugation/filtration. By way of example, a two-step purification protocol can include purification by affinity chromatography (e.g., using heparin affinity resin) followed by polishing on an ion-exchange column. Another illustrative two-step purification protocol can involve ultracentrifugation (e.g., iodixanol density ultracentrifugation) with subsequent chromatography (e.g., affinity chromatography, such as heparin affinity purification).

Filtration and/or centrifugation of the starting material used in the purification process can help to remove some of the bulk impurities, such as for example cell debris and/or cell fragments. Accordingly, in some embodiments, the supernatant is a clarified supernatant. In certain embodiments, filtration and/or centrifugation are performed prior to one or more additional steps of purification, such as for example, chromatographic purification. Various methods for clarifying the supernatant are known in the art. For example, a 0.2 μm filter can be used to clarify the supernatant. In specific embodiments, the clarified supernatant can be treated with Benzonase® prior to one or more additional steps of purification.

Centrifugation techniques for purification of AAV particles can include, for example, density gradient centrifugation, ultracentrifugation, or a combination thereof. An exemplary type of density gradient centrifugation can involve CsCl, which forms a density gradient when subjected to a strong centrifugal field. For example, when the viruses are centrifuged to equilibrium in a CsCl salt, they are separated from contaminants and collected in bands on the basis of their buoyant densities. In some embodiments, purification of AAV particles includes multiple CsCl gradient centrifugation steps. Another exemplary density medium for purification of AAV particles can include iodixanol. In some embodiments, purification of AAV particles can include a density gradient centrifugation (e.g., a discontinuous iodixanol gradient centrifugation) as a pre-purification step, followed by an affinity chromatography virus purification step, such as by a heparinized support matrix chromatography or ion-exchange chromatography.

In some embodiments, purification of AAV particles involves chromatography. For example, the purification of large-scale quantities of AAV particles generally involves some form of chromatography whereby molecules in solution (mobile phase) are separated based on differences in chemical or physical interaction with a stationary material (solid phase or solid support). An exemplary type of chromatography includes, for example, gel filtration (also called size-exclusion chromatography or SEC), which uses a porous resin material to separate molecules based on size (i.e., physical exclusion). Another illustrative type of chromatography is affinity chromatography (also called affinity purification).

In certain embodiments the chromatography involves a chromatographic column. As disclosed herein, various types of chromatographic columns can be used to purify AAV particles. In certain embodiments, the chromatographic column is a monolith. A monolith is a chromatographic column having a single block of a homogenous stationary phase with many interconnected channels. The stationary phase of the monolith can be of various chemistries, allowing the purification of different kinds of biomolecules with different characteristics. However, it is understood that the column need not be a monolith, and that beads, porous particle-based columns and membrane adsorbers can also be used.

Affinity chromatography makes use of specific binding interactions between molecules, such as ligand binding to a target molecule or a specific ionic interaction with a target molecule. An illustrative type of affinity chromatography involves separating viral particles from protein and nucleic acid contaminants based on a reversible interaction between the viral capsid and a specific biological ligand or receptor coupled to a chromatographic matrix. In some embodiments, purification by affinity chromatography can include a negatively charged cellulose affinity medium cellulofine sulfate. An alternative affinity purification approach is based on the recognition of AAV particles (e.g., AAV2 particles) by a monoclonal antibody (e.g., A20), allowing separation of unassembled capsid proteins. Additional illustrative examples for affinity chromatography include heparin affinity.

In some embodiments, affinity chromatography can be specific to the AAV capsid serotype or pseudotype of the AAV particle that is being purified. For example, some AAV serotypes, such as for example AAV1, 4 and 5, bind heparin columns less efficiently. Accordingly, in some embodiments, the affinity matrix for capture of, for example, AAV5 particles can include a sialic acid-rich protein called mucin covalently coupled to CNBr-activated Sepharose. Alternatively, PDGFR-alpha and PDGFR-beta can be used as specific molecules for the capture of, for example, AAV5 particles.

In some embodiments, the chromatography is ion exchange chromatography. Ion exchange chromatography involves the separation of molecules according to the strength of their overall ionic interaction with a solid phase material. Purification by ion-exchange chromatography is based on the net charge of proteins on the exterior of the viral capsid. The net charge of the surface proteins depends on the pH of the exposed amino-acid groups.

One exemplary type of ion exchange chromatography is anion exchange chromatography, which is used to separate molecules based on their net surface charge. Anion exchange chromatography uses a positively charged ion exchange resin with an affinity for molecules having net negative surface charges. It is understood that the examples provided above are intended to be exemplary and are not intended to be exhaustive of the types of chromatography that could be used with the present disclosure.

As provided herein, one exemplary type of purification that can be employed in the process of purifying the AAV particles is affinity chromatography. In some embodiments, the affinity chromatography can involve a particular ligand that is chemically immobilized or “coupled” to a solid support (e.g., affinity support) so that when a complex mixture is passed over the column, those molecules having specific binding affinity to the ligand become bound. In other embodiments, the affinity chromatography can involve ionic interaction based on a specific net surface charge so that the molecules having a specific binding affinity to the solid support based on their net surface charge become bound.

In some embodiments, the affinity chromatography is an ionic exchange chromatography. As described previously, ion exchange chromatography separates molecules according to the strength of their overall ionic interaction with a solid phase material, such as an affinity support. In some embodiments, the ionic exchange chromatography is anion exchange chromatography. Anion exchange chromatography can be used to separate molecules based on their net surface charge. For example, anion exchange chromatography uses a positively charged ion exchange resin with an affinity for molecules having net negative surface charges.

As described above, chromatography generally involves molecules in solution (mobile phase) that are separated based on differences in chemical or physical interaction with a stationary material (solid phase or solid support). The various forms of chromatography can optionally also involve washes to remove the unwanted components from the solid support. Thus, in some embodiments, the methods provided herein involve washing the solid support. After the other sample components are washed away, the bound molecule is stripped from the support (i.e., eluted), resulting in its purification from the original sample.

Elution of the AAV particles can be eluted either by a linear gradient elution or by using a step isocratic elution. Often, a gradient elution may be used to optimize elution conditions. Once the elution profile of the protein of interest has been established and it is known at what ionic strength or pH a protein elutes, a step elution can be used to speed the purification process. Depending on the type of chromatography that is used to purify the AAV particles, the elution conditions involve a competitive ligand, or involve changing pH, ionic strength, or polarity. The target protein can be eluted in a purified and concentrated form. For example, for ion exchange chromatography, the end-product can be eluted in an order depending on their net surface charge. Samples with pI values closer to 7.5 will elute at a lower ionic strength, and samples with very low pI values will elute at a high salt concentration.

In some embodiments, the AAV particles can be eluted using a low pH buffer. In certain embodiments, a high pH buffer is used immediately prior to the use of the low pH buffer, termed “high/low pH buffer.” In specific embodiments, the low pH is about pH 2.5. In some embodiments, the low pH buffer is a citrate buffer, a glycine buffer, or a phosphoric acid buffer. In certain embodiments, the low pH buffer comprises a weak acid.

As provided herein, the addition of ethanol to the elution step can improve the recovery of virus from the ion exchange column. Accordingly, in some embodiments, the elution buffer can include ethanol. In some embodiments, the ethanol can be about 5% to about 40% ethanol. In some embodiments, the ethanol can be about 10% to about 30% ethanol. In some embodiments, the ethanol can be about 15% to about 25% ethanol. In specific embodiments, the ethanol can be about 20% ethanol.

Elution performed using a low pH buffer often requires the elution buffer to be immediately neutralized. Thus, in some embodiments, the elution further includes neutralizing the pH of the buffer. In specific embodiments, neutralizing comprises adding Bis-Tris-Propane (BTP). In certain embodiments, neutralizing comprises adding Tris. Because many chromatographic elution buffers used for Ad or AAV purification procedures are not suitable for in vivo manipulations, additional purification steps such as dialysis or concentration may be necessary. Therefore, in some embodiments, the purification also includes dialysis and/or concentration of the AAV viral particles.

Thus, in some embodiments, provided herein is a method for purifying AAV particles that includes (a) incubating a supernatant comprising AAV particles with a solid support for a sufficient amount of time to bind the AAV particles; (b) contacting the supernatant comprising AAV particles with a composition comprising a chromatin-DNA nuclease; and (c) eluting the purified AAV particles. It is understood that contacting with the chromatin-DNA nuclease can also be performed before the binding of the AAV particles. Accordingly, in some embodiments, provided herein is a method for purifying adeno-associated viral (AAV) particles that includes (a) contacting the supernatant comprising AAV particles with a composition comprising a chromatin-DNA nuclease; (b) incubating a supernatant comprising AAV particles with a solid support for a sufficient amount of time to bind the AAV particles; and (c) eluting the purified AAV particles. Similarly, it is also understood that the contacting with the chromatin-DNA nuclease need not be combined in the setting of a solid support and can be combined with any AAV particle purification technique known in the art. In certain embodiments, the purified AAV particles are subjected to one or more additional purifications to polish the AAV particles. For example the particles can be purified by affinity chromatography and then polished by a different type of chromatography, such as anion exchange chromatography.

In some embodiments, the method further includes washing the solid support before eluting the sample. As described herein, the washing away of non-bound sample components from the support can be performed using appropriate buffers that maintain the binding interaction between target and ligand. The washing can remove some unbound contaminants. In some embodiments, nonspecific binding interactions can be minimized by adding low levels of detergent or by moderate adjustments to salt concentration in the binding and/or wash buffer.

As provided herein, in some embodiments, the purity of the AAV particles can be increased by washing with a high pH. For example, the bulk harvest can be purified by affinity chromatography and then washed with a high pH buffer to remove impurities. In some embodiments, the high pH wash is followed by on-column enzyme treatment with benozonase and/or a chromatin-DNA nuclease. In certain embodiments, the high pH wash buffer is greater than pH 9. In some embodiments, the high pH wash buffer is between pH 9.5 and pH 10.9. In other embodiments, the high pH wash buffer is pH 9.5. In some embodiments, the high pH wash buffer is pH 10.2. In some embodiments, the high pH wash buffer is pH 10.3. In some embodiments, the high pH wash buffer is pH 10.4.

In some embodiments, the chromatin-DNA nuclease is micrococcal nuclease (MNase) (EC 3.1.31.1). MNase isolated from Staphylococcus aureus is a phosphodiesterase with non-specific endo-exonuclease activity capable of digesting nucleic acids (DNA and/or RNA). MNase digests exposed nucleic acids within the linker region connecting two nucleosomes until it reaches an obstruction (nucleosome or other nucleic acid binding protein). MNase can be suitable for removing nucleic acids from cell lysates, releasing chromatin-bound proteins, whereas DNase preferentially cleaves nucleosome-depleted or “free” DNA. MNase digests double-stranded, single-stranded, circular and linear nucleic acids. In some embodiments, the concentration of the MNase in the supernatant is greater than 2.5 units/mL (U/mL). In certain embodiments, the concentration of the MNase in the supernatant is greater than 10 units/mL. In specific embodiments, the concentration of the MNase in the supernatant is about 30 units/mL to about 100 units/mL. In more specific embodiments, the concentration of the MNase in the supernatant is about 60 units/mL. In some embodiments, the MNase is a polypeptide having the activity of a MNase. In one embodiment, the MNase is present in a sufficient amount to digest chromatin associated with an AAV particle.

In one embodiment, the MNase is present in a sufficient amount to reduce AAV particle impurities. In one embodiment, the AAV particle impurities comprise one or more of a host-cell DNA, a host-cell protein, a chromatin-associated DNA, and a DNA binding protein. In one embodiment, the AAV particle impurities comprise macroscopic and microscopic impurities. In one embodiment, the DNA binding protein comprises a histone.

In one embodiment, the MNase is present in an amount sufficient to increase a viral titer of AAV particles. In one embodiment, the viral titer comprises a physical viral titer, a functional viral titer, or both. In one embodiment, the viral titer comprises a physical titer. In one embodiment, the viral titer comprises a functional titer. In one embodiment, the viral titer is increased about 2 fold to about 100 fold. In one embodiment, the viral titer is increased about 2 fold or greater. In one embodiment, the viral titer is increased about 3 fold or greater. In one embodiment, the viral titer is increased about 7 fold or greater. In one embodiment, the viral titer is increased about 80 fold or greater.

In one embodiment, the MNase is present in a sufficient amount to increase a viral titer ratio of a product AAV particle fraction to a post-product AAV particle fraction.

In one embodiment, the viral titer ratio is increased about 2 fold or greater. In one embodiment, the viral titer ratio is increased about 5 fold or greater. In one embodiment, the viral titer ratio is increased about 10 fold or greater. In one embodiment, the viral titer ratio is increased about 25 fold or greater.

In one embodiment, the MNase is present in an amount sufficient to increase a full-to-empty capsid ratio. In one embodiment, the full-to-empty capsid ratio is greater than about 50%. In one embodiment, the full-to-empty capsid ratio is greater than about 60%. In one embodiment, the full-to-empty capsid ratio is greater than about 70%.

In one embodiment, the MNase is present in an amount sufficient to decrease a AAV particle post-product fraction, as measured by absorbance at 260 nm. In one embodiment, the MNase is present in an amount sufficient to decrease a AAV particle post-product fraction, as measured by absorbance at 280 nm.

In one embodiment, the MNase is present in an amount sufficient to produce purified AAV particles comprising a melting temperature (Tm) within less than about 10° C. of an aggregation temperature (Tagg), as measured by dynamic light scatter (DLS).

In one embodiment, the MNase is present in an amount sufficient to produce purified AAV particles comprising a melting temperature (Tm) within less than about 5° C. of an aggregation temperature (Tagg), as measured by dynamic light scatter (DLS).

In one embodiment, the MNase is present in an amount sufficient to produce purified AAV particles comprising a melting temperature (Tm) within less than about 2° C. of an aggregation temperature (Tagg), as measured by dynamic light scatter (DLS).

Determination of the length of time necessary for the chromatin-DNA nuclease to release DNA binding proteins extra-virally complexed to AAV particles and free nucleic acids are within the skillset of a person skilled in the art. In certain embodiments, the incubation is for about 10 minutes to about 1 hour. In some embodiments, the incubation is for about 20 minutes to about 40 minutes. In specific embodiments, the incubation is for about 30 minutes.

In some embodiments of the methods provided herein, the composition that includes a chromatin-DNA nuclease can also include a Benzonase® nuclease (an endonuclease from Serratia marcescens; Enzyme Commission (EC) Number 3.1.30.2). Benzonase® is a promiscuous endonuclease that can degrade accessible DNA and RNA (e.g., non-chromatin DNA). It attacks and degrades all forms of DNA and RNA (e.g., single stranded, double stranded, linear and circular) and is effective over a wide range of operating conditions. For example, it can digest native or heat-denatured DNA and RNA.

The addition of Benzonase® can help to remove nuclease-sensitive nucleic acids present in the crude sample, such as residual nucleic acids from the host production cell. Although the addition of Benzonase® can be included to digest free nucleic acid, such as to reduce viscosity in protein samples, by itself it is insufficient to release DNA binding proteins extra-virally complexed to AAV particles. In some embodiments, the Benzonase® and the chromatin-DNA nuclease are incubated together. In specific embodiments, the Benzonase® and the chromatin-DNA nuclease are incubated together after affinity exchange chromatography. However, it is understood that the Benzonase® treatment need not be performed simultaneously with a chromatin-DNA nuclease. For example, in some embodiments, the Benzonase® is added to the bulk harvest before purification. It is further understood that any Benzonase® product is suitable with the present disclosure.

Although the present disclosure describes the use of Benzonase® as an exemplary endonuclease in certain embodiments, it is understood that any nuclease capable of reducing residual host cell DNA or a polypeptide having the activity of a nuclease capable of reducing residual host cell DNA can be used. Such alternative nucleases can include, for example, a cryonase (a recombinant endonuclease originating from a psychrophile, Shewanella sp.), a salt active nuclease (SAN), or DNase I™.

As provided herein, contacting sample containing AAV particles with MNase can remove chromatin-associated DNA, host cell proteins, and improve the overall yield of the AAV particles, relative to non-MNase treated samples. Therefore, in some embodiments, the purified AAV particles prepared using the methods provided herein are substantially free of chromatin-associated DNA, when compared to non-MNase contacted purified AAV particles. In certain embodiments, the purified AAV particles are substantially free of host cell proteins, when compared to non-MNase contacted purified AAV particles. In some embodiments, the AAV particles have an increased yield, when compared to non-MNase contacted purified AAV particles.

As provided herein, the present disclosure provides that the use of a chromatin-DNA nuclease in the purification of AAV particles can be used to increase viral titers. Thus, in some embodiments, provided herein in a method for increasing titers of AAV particles, where the method includes: (a) contacting a supernatant comprising AAV particles with a composition comprising a chromatin-DNA nuclease; and (b) purifying the AAV particles.

In some embodiments, the increased viral titer can include an increase in the total genome copies, as well as the genome copies per cell (i.e., physical titer). In other embodiments, the increased viral titer can include an increase in the functional titer. In further embodiments, the increased viral titer can include both of an increase in the physical titer and the functional titer.

Physical titers can be determined using non-functional methods, such as for example, an ELISA, a measurement of viral genomic RNA (e.g., by qRT-PCR, or Northern blotting). Functional titer measures how much virus gets into a target cell, and can include assessment of the number of colony forming units following antibiotic selection if the vector contains an antibiotic resistance gene, or, if the vector contains a fluorescent protein, flow cytometry or immunofluorescence analysis of the target cells. Alternatively, if the vector does not express a fluorescent protein, determining the number of integrated proviral DNA copies per cell by qPCR provides a fast and easy method for assessing functional titer.

An increase in the titer can include a fold-change of any integer greater than 1.0, relative to a supernatant comprising AAV particles not contacted with a composition comprising a chromatin-DNA nuclease. In some embodiments, the fold change is greater than 1.5 fold, greater than 2.0 fold, greater than 5.0 fold, greater than 10 fold, greater than 50 fold, or greater than 100 fold. It is understood that functional titer will generally be lower than physical titer, usually by a factor of 10 to 100-fold. Thus, in some embodiments, the increase in physical titer is greater than 1.5 fold, greater than 2.0 fold, greater than 5.0 fold, greater than 10 fold, greater than 50 fold, or greater than 100 fold. In specific embodiments, the increase in physical titer is greater than 1.5 fold. In some embodiments, the increase in physical titer is greater than 2.0 fold. In some embodiments, the increase in physical titer is greater than 5.0 fold. In some embodiments, the increase in physical titer is greater than 10 fold. In some embodiments, the increase in physical titer is greater than 50 fold. In some embodiments, the increase in physical titer is greater than 100 fold. In other embodiments, the increase in functional titer is greater than 1.5 fold, greater than 2.0 fold, greater than 5.0 fold, greater than 10 fold, greater than 50 fold, or greater than 100 fold. In some embodiments, the increase in functional titer is greater than 1.5 fold. In some embodiments, the increase in functional titer is greater than 1.5 fold. In some embodiments, the increase in functional titer is greater than 2.0 fold. In some embodiments, the increase in functional titer is greater than 5.0 fold. In some embodiments, the increase in functional titer is greater than 10 fold. In some embodiments, the increase in functional titer is greater than 50 fold. In some embodiments, the increase in functional titer is greater than 100 fold.

As provided herein, the present disclosure demonstrates that the addition of a chromatin-DNA nuclease (e.g., MNase) to the AAV purification process can increase the viral titer of the product fraction, and decrease the viral titer of the post-product fraction. Accordingly, the increase in viral titer can also be described as a ratio of the AAV product fraction compared to the AAV post-product fraction. Thus, in some aspects, the increased viral titer is an increased viral titer ratio of a product AAV particle fraction to a post-product AAV particle fraction, relative to AAV particles not contacted with a chromatin-DNA nuclease (e.g., MNase). In some embodiments, the increase in viral titer ratio is greater than 1.5 fold, greater than 2.0 fold, greater than 5.0 fold, greater than 10 fold, greater than 50 fold, or greater than 100 fold. In specific embodiments, the increase in viral titer ratio is about 2 fold or greater. In other embodiments, the increase in viral titer ratio is about 5 fold or greater. In still other embodiments, the increase in viral titer ratio is about 10 fold or greater. In other embodiments, the increase in viral titer ratio is about 25 fold or greater.

As described herein, the methods of the present disclosure relate to the production of highly pure AAV particles that are substantially free of an impurity. Various techniques are known in the art for measuring AAV physical properties (e.g., AAV particle size). For example, exemplary techniques for measuring particle size and aggregation include dynamic light scattering (DLS), static light scattering (SLS), DLS and SLS, and transmission electron microscopy (TEM) (see, e.g., Stetefeld J, et al., Biophys Rev. 2016; 8(4):409-427).

DLS is a well-established analytical technique in the field of AAV development. Its primary use is to test for aggregate formation. Due to its high sensitivity towards large species, even small impurities caused by aggregation can be detected. It is also possible to combine DLS with SLS. In DLS, the hydrodynamic size and size distribution of particles in solution can be obtained. It may be of interest to examine this measurement as a function of time and temperature. For example, although at low temperatures a protein may be stable and show repeatable size (and scattering intensity) measurements, typically at some elevated temperature (Tagg), protein molecules will show a tendency to oligomerize or aggregate. The temperature at which this occurs will depend on the protein itself, plus the buffer composition. DLS or the combination of DLS and SLS therefore allows the instrument user to screen the melting (Tm), aggregation (Tagg) and onset temperatures (Tonset). Samples that have a Tagg that is near similar to the Tm are indicative of highly pure AAV particles that are substantially free of an impurity.

Accordingly, in some embodiments of the methods described herein, the purified AAV particles have a Tm within less than about 10° C. of Tagg, as measured by DLS. In certain embodiments, the purified AAV particles have a Tm within less than about 5° C. of Tagg, as measured by DLS. In further embodiments, the purified AAV particles have a Tm within less than 2° C. of Tagg, as measured by DLS.

The present disclosure also demonstrates the addition on MNase to the purification process of AAV particles can increase the amount of full capsid recovered. In some aspects of the present disclosure, the purified AAV particles comprise a full-to-empty capsid ratio of greater than about 50%. In other embodiments, the purified AAV particles comprise a full-to-empty capsid ratio of greater than about 60%. In still further embodiments, the purified AAV particles comprise a full-to-empty capsid ratio of greater than about 70%.

The present disclosure also provides a composition of AAV particles produced by any of the methods provided herein. In some embodiments, the composition is substantially free of a visible or subvisible precipitate (i.e., macroscopic or microscopic), when compared to a composition not contacted with a chromatin-DNA nuclease. In certain embodiments, the composition is substantially free of chromatin-associated DNA, when compared to a composition not contacted with a chromatin-DNA nuclease. In some embodiments, the composition is substantially free of host cell proteins, when compared to a composition not contacted with a chromatin-DNA nuclease. In some embodiments, the composition is substantially free of DNA binding proteins and/or chromatin associated proteins, when compared to a composition not contacted with a chromatin-DNA nuclease. In specific embodiments, the DNA binding protein comprises a histone (e.g., H1, H2A, H2B, H3, and H4), and the composition is substantially free of histones, when compared to a composition not contacted with a chromatin-DNA nuclease. In some embodiments, the composition has an increased viral titer. In specific embodiments, the viral titer comprises a physical titer. In other embodiments, the viral titer comprises a functional titer. In specific embodiments, the composition has a reduced post-product peak as measured by an anion exchange chromatogram, when compared to a composition not contacted with a chromatin-DNA nuclease. In more specific embodiments, the chromatin-DNA nuclease is MNase.

Also provided herein is a composition, comprising (a) a supernatant comprising AAV particles; and (b) a chromatin-DNA nuclease. In some embodiments, the composition further includes Benzonase®. In specific embodiments, the chromatin-DNA nuclease is MNase.

The present disclosure also provides a composition for use in producing an AAV particle that is substantially free of chromatin-associated DNA, the composition comprising: (a) a supernatant comprising AAV particles; and (b) a chromatin-DNA nuclease. In some embodiments, the composition further includes Benzonase®. In specific embodiments, the chromatin-DNA nuclease is MNase.

Determining the concentration of the MNase suitable for the composition is within the skillset of a person skilled in the art. In some embodiments, the concentration of the MNase is greater than 2.5 units/mL. In certain embodiments, the concentration of the MNase is greater than 10 units/mL. In specific embodiments, the concentration of the MNase is about 30 units/mL to about 100 units/mL. In some specific embodiments, the concentration of the MNase in the supernatant is about 60 units/mL.

Also provided herein, is a kit comprising, (a) Benzonase®; and (b) a chromatin-DNA nuclease. In some embodiments, the chromatin-DNA nuclease is MNase. In certain embodiments, the MNase is present in a sufficient amount to enhance AAV particle purification. In some embodiments, the MNase is present in a sufficient amount to release a chromatin-bound protein.

Various systems and particle production platforms are currently in use for the making of AAV particles and are known in the art, each of which is suitable for use in the purification methods, compositions, and systems described herein. Exemplary methods and systems for the generation of AAV particles at large scale can involve, for example, plasmid DNA transfection in mammalian cells, Ad infection of stable mammalian cell lines, infection of mammalian cells with recombinant herpes simplex viruses (rHSVs), and infection of insect cells with recombinant baculoviruses (see, e.g., Penaud-Budloo M. et al., Mol Ther Methods Clin Dev. 2018 Jan. 8; 8:166-180).

An exemplary method or system for the production of AAV particles is, for example, the plasmid transfection of human embryonic HEK293 cells. For example, HEK293 cells can be simultaneously transfected with a plasmid containing the gene of interest and one or two helper plasmids, using either inorganic compounds (e.g. calcium phosphate) or organic compounds (e.g. polyethyleneimine (PEI)), or non-chemical (e.g. electroporation). The helper plasmid(s) allow the expression of the four Rep proteins (Rep78, Rep68, Rep52, Rep40), the three AAV structural proteins (VP1, VP2, and VP3), the AAP, and the adenoviral auxiliary functions E2A, E4, and VA RNA. The additional adenoviral E1A/E1B co-factors necessary for AAV replication can be expressed in HEK293 producer cells. The plasmids can be produced by conventional techniques in E coli using bacterial origin and antibiotic-resistance gene or by minicircle (MC) technology. The producer cells, such as HEK293 producer cells, can be adherent or suspension cultures.

Another illustrative method or system for production involves infection of mammalian cells with rHSV vectors. Cells, such as the hamster BHK21 cell line or HEK293 and derivatives, can be infected with two rHSVs, one carrying the gene of interest bracketed by AAV ITR (rHSV-AAV) and the second with the AAV rep and cap ORFs of the desired serotype (rHSV-repcap) for the production of AAV particles.

Stable producer cell lines for AAV particle production offer a further illustrative system for the production of AAV particles. For example, stable producer cell lines can be derived from a cell line (e.g., HEK293 cells, HeLa cells, or a derivative) and engineered by introducing either the AAV rep and cap genes (packaging cell lines) and/or the AAV genome (e.g., rAAV genome) to be produced (producer cells). Another illustrative stable cell line for the production of AAV particles includes a stable cell line that incorporates the usually-toxic AAV replication (rep) gene as well as an AAV capsid (cap) gene and a transgene (see, e.g., U.S. Application No. 62/877,508, which is disclosed herein in its entirety).

It is further understood that the producer cell line need not be a mammalian cell line, and that non-mammalian cells, such as insect cells, and yeast can be used for the production. An illustrative example of a non-mammalian platform suitable for production of AAV particles includes the baculovirus-Sf9 insect-cell platform. The non-mammalian cell line suitable for production of AAV particles can be generated using transfection methods or as a stable cell line (see, e.g., Mietzsch M. et al. Hum. Gene Ther. 2014; 25: 212-222; Mietzsch M. et al. Hum. Gene Ther. Methods. 2017; 28: 15-22). The examples of AAV particle production platforms described above are understood to be illustrative, and not intended to be limiting, and that any of the various production platforms can be combined with the purification methods and compositions described herein.

As used herein, a “vector” is a nucleic acid molecule used to carry genetic material into a cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

In some embodiments of the application, a vector is a non-viral vector. Examples of non-viral vectors include, but are not limited to, DNA plasmids, bacterial artificial chromosomes, yeast artificial chromosomes, bacteriophages, etc. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of DNA plasmids suitable that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNA™ or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pUC, pcDNA™, pcDNA3™, pVAX, pVAX-1, ADVAX, NTC8454, etc. For example, the vector can be based on pUC57, containing a pUC origin of replication and ampicillin resistance gene. It can further comprise a mammalian puromycin resistance gene cassette constructed from the Herpes virus thymidine kinase gene promoter, the puromycin N-acetyl transferase coding region, and a polyadenylation signal from bovine growth hormone gene. The vector can also comprise an Epstein Barr Virus (EBV) OriP replication origin fragment, which represents a composite of the ‘Dyad Symmetry’ region and the ‘Family of Repeats’ region of EBV.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, etc. The vector can also be a non-viral vector.

An illustrative viral vector is an adenovirus vector, e.g., a recombinant adenovirus vector. As used herein, the terms “recombinant adenovirus vector” and “recombinant adenoviral vector” and “recombinant adenoviral particles” are used interchangeably and refer to a genetically-engineered adenovirus that is designed to insert a polynucleotide of interest into a eukaryotic cell, such that the polynucleotide is subsequently expressed. Examples of adenoviruses that can be used as a viral vector of the invention include those having, or derived from, the serotypes Ad2, Ad5, Ad11, Ad12, Ad24, Ad26, Ad34, Ad35, Ad40, Ad48, Ad49, Ad50, Ad52 (e.g., RhAd52), and Pan9 (also known as AdC68); these vectors can be derived from, for example, human, chimpanzee (e.g., ChAd1, ChAd3, ChAd7, ChAd8, ChAd21, ChAd22, ChAd23, ChAd24, ChAd25, ChAd26, ChAd27.1, ChAd28.1, ChAd29, ChAd30, ChAd31.1, ChAd32, ChAd33, ChAd34, ChAd35.1, ChAd36, ChAd37.2, ChAd39, ChAd40.1, ChAd41.1, ChAd42.1, ChAd43, ChAd44, ChAd45, ChAd46, ChAd48, ChAd49, ChAd49, ChAd50, ChAd67, or SA7P), or rhesus adenoviruses (e.g., rhAd51, rhAd52, or rhAd53). A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd).

Preferably, an adenovirus vector is a recombinant human adenovirus vector, for instance a recombinant human adenovirus serotype 5, or any one of recombinant human adenovirus serotype 26, 4, 35, 7, 48, etc. A recombinant viral vector useful for the application can be prepared using methods known in the art in view of the present disclosure. For example, in view of the degeneracy of the genetic code, several nucleic acid sequences can be designed that encode the same polypeptide. A polynucleotide encoding a protein of interest can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

A non-naturally occurring nucleic acid molecule or a vector can comprise one or more expression cassettes. An “expression cassette” is part of a nucleic acid molecule or vector that directs the cellular machinery to make RNA and protein. An expression cassette can comprise a promoter sequence, an open reading frame, a 3′-untranslated region (UTR) optionally comprising a polyadenylation signal. An open reading frame (ORF) is a reading frame that contains a coding sequence of a protein of interest (e.g., Rep, Cap, recombinase or a recombinant protein of interest) from a start codon to a stop codon. Regulatory elements of the expression cassette can be operably linked to a polynucleotide sequence encoding a protein of interest.

A non-naturally occurring nucleic acid molecule or a vector of the application can contain a variety of regulatory sequences. A s used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e., mRNA) into the host cell or organism. Regulatory elements include, but are not limited to, a promoter, an enhancer, a polyadenylation signal, translation stop codon, a ribosome binding element, a transcription terminator, selection markers, origin of replication, etc.

A non-naturally occurring nucleic acid molecule or a vector can comprise a promoter sequence, preferably within an expression cassette, to control expression of a protein of interest. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding a protein of interest within an expression cassette.

Examples promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein. A promoter can also be a tissue specific promoter, such as a muscle or skin specific promoter, natural or synthetic. Preferably, a promoter is a strong eukaryotic promoter, such as a cytomegalovirus (CMV) promoter (nt −672 to +15), EF1-alpha promoter, herpes virus thymidine kinase gene promoter, etc.

A non-naturally occurring nucleic acid molecule or a vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., Rep, Cap, recombinase) within an expression cassette of the vector. Enhancer sequences are regulatory DNA sequences that, when bound by transcription factors, enhance the transcription of an associated gene. An enhancer sequence is preferably downstream of a promoter sequence and can be downstream or upstream of a coding sequence within an expression cassette of the vector.

Any polyadenylation signal known to those skilled in the art in view of the present disclosure can be used. For example, the polyadenylation signal can be a SV40 polyadenylation signal, (AAV2 polyadenylation signal (bp 4411-4466, NC_001401), a polyadenylation signal from the Herpes Simplex Virus Thymidine Kinase Gene, LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadenylation signal, the polyadenylation signal of AAV2 having nucleotide numbers 4411 to 4466 of the nucleotide sequence of GenBank accession number NC_001401, or a SV40 polyadenylation signal.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit p-globin intron, or any combination thereof.

Preferably, an enhancer sequence comprises a P5 promoter of an AAV. The P5 promoter is part of a cis-acting Rep-dependent element (CARE) inside the coding sequence of the rep gene. CARE was shown to augment the replication and encapsidation when present in cis. CARE is also important for amplification of chromosomally integrated rep genes (if AAV ITRs are not present) as in some AAV particle producer cell lines. While not wishing to be bound by theories, it is believed that a P5 promoter placed downstream of a cap coding sequence potentially act as an enhancer to increase Cap expression, thus AAV particle yields, and that it also provides enhancer activity for amplifying genes integrated into a chromosome.

A non-naturally occurring nucleic acid molecule or a vector, such as a DNA plasmid, can also include a bacterial origin of replication and an antibiotic resistance expression cassette for selection and maintenance of the plasmid in bacterial cells, e.g., E. coli. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Vectors for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan), ampicillin resistance gene (Amp), and tetracycline resistance gene (Tetr), as well as genes conferring resistance to chloramphenicol, bleomycin, spectinomycin, carbenicillin, etc.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EMBODIMENTS

This invention provides the following non-limiting embodiments.

In one set of embodiments, provided are:

A1. A method for purifying adeno-associated viral (AAV) particles, said method comprising:
(a) contacting a supernatant comprising AAV particles with a composition comprising a chromatin-DNA nuclease; and
(b) purifying the AAV particles.

A2. The method of embodiment A1, wherein purifying comprises centrifugation, chromatography, filtration, or a combination thereof.

A3. The method of embodiment A2, wherein centrifugation comprises density gradient centrifugation, ultracentrifugation, or a combination thereof.

A4. The method of embodiment A2, wherein chromatography comprises affinity chromatography, ion exchange chromatography, size exclusion chromatography, hydrophobic interaction chromatography, or a combination thereof.

A5. The method of embodiment A4, further comprising incubating the supernatant comprising AAV particles with a solid support for a sufficient amount of time to bind the AAV particles.

A6. The method of embodiment A5, further comprising washing the solid support.

A7. The method of embodiment A6, wherein the washing comprises a high pH buffer.

A8. The method of embodiment A7, wherein the high pH buffer is greater than pH 9.0.

A9. The method of embodiment A7, wherein the high pH buffer is between pH 9.0 and pH 11.

A10. The method of embodiment A7, wherein the high pH buffer is about pH 9.5.

A11. The method of embodiment A7, wherein the high pH buffer is about pH 10.2.

A12. The method of embodiment A7, wherein the high pH buffer is about pH 10.3.

A13. The method of embodiment A7, wherein the high pH buffer is about pH 10.4.

A14. The method of any one of embodiments A4 to A13, wherein the method comprises one or more affinity chromatography purifications.

A15 The method of embodiment A14, wherein the affinity chromatography comprises ion exchange chromatography.

A16. The method of embodiment A15, wherein the ion exchange chromatography comprises anion exchange chromatography.

A17. The method of any one of embodiments A1 to A16, wherein the supernatant is a clarified supernatant.

A18. The method of any one of embodiments A1 to A17, wherein the composition of step (a) further comprises Benzonase®.

A19. The method of any one of embodiments A1 to A18, wherein the incubation is for about 10 minutes to about 1 hour.

A20. The method of embodiment A19, wherein the incubation is for about 20 minutes to about 40 minutes.

A21. The method of embodiment A19 or A20, wherein the incubation is for about 30 minutes.

A22. The method of any one of embodiments A1 to A21, wherein the chromatin-DNA nuclease is micrococcal nuclease (MNase).

A23. The method of embodiment A22, wherein the concentration of the MNase in the supernatant is greater than 2.5 units/mL.

A24. The method of embodiment A23, wherein the concentration of the MNase in the supernatant is greater than 10 units/mL.

A25. The method of embodiment A23 or A24, wherein the concentration of the MNase in the supernatant is about 30 units/mL to about 100 units/mL.

A26. The method of any one of embodiments A23 to A25, wherein the concentration of the MNase in the supernatant is about 60 units/mL.

A27. The method of any one of embodiments A22 to A26, wherein the MNase is incubated with the solid support containing bound AAV particles.

A28. The method of any one of embodiments A5 to A27, wherein the AAV particles are eluted from the solid support using a low pH buffer.

A29. The method of embodiment A28, further comprising a high pH buffer prior to the low pH buffer.

A30. The method of embodiment A28 or A29, wherein the low pH buffer is less than about pH 3.0.

A31. The method of embodiment A30, wherein the low pH buffer is about pH 1.5 to about pH 2.5.

A32. The method of embodiment A30, wherein the low pH buffer is about pH 1.5.

A33. The method of embodiment A30, wherein the low pH buffer is about pH 2.5.

A34. The method of any one of embodiments A28 to A33, wherein the low pH buffer is a citrate buffer, glycine buffer, or a phosphoric acid buffer.

A35. The method of embodiment A34, wherein the low pH buffer is a citrate buffer.

A36. The method of embodiment A34, wherein the low pH buffer is a phosphoric acid buffer.

A37. The method of any one of embodiments A14 to A36, wherein the affinity chromatography purification comprises two affinity chromatography purifications.

A38. The method of embodiment A37, wherein the method comprises an affinity chromatography purification followed by an anion-exchange chromatography.

A39. The method of any one of embodiments A28 to A38, further comprising neutralizing the pH of the low pH buffer.

A40. The method of embodiment A39, wherein neutralizing comprises adding Bis-Tris-Propane (BTP) or a Tris buffer.

A41. The method of any one of embodiments A28 to A40, further comprising about 5% to about 40% ethanol.

A42. The method of embodiment A41, comprising about 10% to about 30% ethanol.

A43. The method of embodiment A41 or A42, comprising about 15% to about 25% ethanol.

A44. The method of any one of embodiments A41 to A43, comprising about 20% ethanol.

A45. The method of any one of embodiments A1 to A44, wherein the purified AAV particles are substantially free of chromatin-associated DNA, when compared to non-MNase contacted purified AAV particles.

A46. The method of any one of embodiments A1 to A45, wherein the purified AAV particles are substantially free of host-cell DNA, when compared to non-MNase contacted purified AAV particles.

A47. The method of embodiment A46, wherein the host-cell DNA concentration is less than 2 ng/mL.

A48. The method of embodiment A46, wherein the host-cell DNA concentration is less than 1.5 ng/mL.

A49. The method of embodiment A46, wherein the host-cell DNA concentration is less than 1 ng/mL.

A50. The method of any one of embodiments A1 to A49, wherein the purified AAV particles are substantially free of host cell proteins, when compared to non-MNase contacted purified AAV particles.

A51. The method of embodiment A50, wherein the purified AAV particles are substantially free of a DNA binding protein, when compared to non-MNase contacted purified AAV particles.

A52. The method of embodiment A51, wherein the DNA binding protein comprises a histone.

A53. The method of any one of embodiments A1 to A52, wherein the purified AAV particles are substantially free of macroscopic and microscopic impurities.

A54. The method of any one of embodiments A1 to A53, wherein the purified AAV particles have an increased viral titer, when compared to non-MNase contacted purified AAV particles.

A55. The method of embodiment A54, wherein the viral titer comprises a physical titer.

A56. The method of embodiment A54, wherein the viral titer comprises a functional titer.

A57. The method of any one of embodiments A54 to A56, wherein the viral titer is increased about 2 fold to about 100 fold.

A58. The method of any one of embodiments A54 to A56, wherein the viral titer is increased about 2 fold or greater.

A59. The method of any one of embodiments A54 to A56, wherein the viral titer is increased about 3 fold or greater.

A60. The method of any one of embodiments A54 to A56, wherein the viral titer is increased about 7 fold or greater.

A61. The method of any one of embodiments A54 to A56, wherein the viral titer is increased about 80 fold or greater.

A62. The method of any one of embodiments A1 to A61, wherein the purified AAV particles comprise an increased viral titer ratio of a product AAV particle fraction to a post-product AAV particle fraction, when compared to non-MNase contacted purified AAV particles.

A63. The method of embodiment A62, wherein the viral titer ratio is increased about 2 fold or greater.

A64. The method of embodiment A62, wherein the viral titer ratio is increased about 5 fold or greater.

A65. The method of embodiment A62, wherein the viral titer ratio is increased about 10 fold or greater.

A66. The method of embodiment A62, wherein the viral titer ratio is increased about 25 fold or greater.

A67. The method of any one of embodiments A1 to A66, wherein the purified AAV particles have a melting temperature (Tm) within less than about 10° C. of an aggregation temperature (Tagg), as measured by dynamic light scatter (DLS).

A68. The method of embodiment A67, wherein the purified AAV particles have a Tm within less than about 5° C. of an aggregation temperature (Tagg), as measured by dynamic light scatter (DLS).

A69. The method of embodiment A67, wherein the purified AAV particles have a melting temperature (Tm) within less than 2° C. of an aggregation temperature (Tagg), as measured by dynamic light scatter (DLS).

A70. The method of any one of embodiments A1 to A69, wherein the purified AAV particles comprise a full-to-empty capsid ratio of greater than about 50%.

A71. The method of embodiment A70, wherein the purified AAV particles comprise a full-to-empty capsid ratio of greater than about 60%.

A72. The method of embodiment A70, wherein the purified AAV particles comprise a full-to-empty capsid ratio of greater than about 70%.

A73. The method of any one of embodiments A1 to A72, wherein the purified AAV particles comprise a AAV particle post-product fraction with a reduced absorbance at 260 nm, when compared to non-MNase contacted purified AAV particles.

A74. The method of any one of embodiments A1 to A73, wherein the purified AAV particles comprise a AAV particle post-product fraction with a reduced absorbance at 280 nm, when compared to non-MNase contacted purified AAV particles.

In another set of embodiments, provided are:

B1. A method for increasing a viral titer of AAV particles, said method comprising:

(a) contacting a supernatant comprising AAV particles with a composition comprising a chromatin-DNA nuclease; and

(b) purifying the AAV particles.

B2. The method of embodiment B1, wherein the viral titer comprises a physical viral titer, a functional viral titer, or both.

B3. The method of embodiment B1, wherein the viral titer comprises a physical viral titer.

B4. The method of embodiment B1, wherein the viral titer comprises a functional viral titer.

B5. The method of embodiment B1, wherein purifying comprises centrifugation, chromatography, filtration, or a combination thereof.

B6. The method of embodiment B5, wherein centrifugation comprises density gradient centrifugation, ultracentrifugation, or a combination thereof.

B7. The method of embodiment B5, wherein chromatography comprises affinity chromatography, ion exchange chromatography, size exclusion chromatography, hydrophobic interaction chromatography, or a combination thereof.

B8. The method of embodiment B7, further comprising incubating the supernatant comprising AAV particles with a solid support for a sufficient amount of time to bind the AAV particles.

B9. The method of embodiment B8, further comprising washing the solid support.

B10. The method of embodiment B9, wherein the washing comprises a high pH buffer.

B11. The method of embodiment B10, wherein the high pH buffer is greater than pH 9.0.

B12. The method of embodiment B10, wherein the high pH buffer is between pH 9.0 and pH 11.

B13. The method of embodiment B10, wherein the high pH buffer is about pH 9.5.

B14. The method of embodiment B10, wherein the high pH buffer is about pH 10.2.

B15. The method of embodiment B10, wherein the high pH buffer is about pH 10.3.

B16. The method of embodiment B10, wherein the high pH buffer is about pH 10.4.

B17. The method of any one of embodiments B7 to B16, wherein the method comprises one or more affinity chromatography purifications.

B18. The method of embodiment B17, wherein the affinity chromatography comprises ion exchange chromatography.

B19. The method of embodiment B18, wherein the ion exchange chromatography comprises anion exchange chromatography.

B20. The method of any one of embodiments B1 to B19, wherein the supernatant is a clarified supernatant.

B21. The method of any one of embodiments B1 to B20, wherein the composition of step (a) further comprises Benzonase®.

B22. The method of any one of embodiments B1 to B21, wherein the incubation is for about 10 minutes to about 1 hour.

B23. The method of embodiment B22, wherein the incubation is for about 20 minutes to about 40 minutes.

B24. The method of embodiment B22 or B23, wherein the incubation is for about 30 minutes.

B25. The method of any one of embodiments B1 to B24, wherein the chromatin-DNA nuclease is micrococcal nuclease (MNase).

B26. The method of embodiment B25, wherein the concentration of the MNase in the supernatant is greater than 2.5 units/mL.

B27. The method of embodiment B26, wherein the concentration of the MNase in the supernatant is greater than 10 units/mL.

B28. The method of embodiment B26 or B27, wherein the concentration of the MNase in the supernatant is about 30 units/mL to about 100 units/mL.

B29. The method of any one of embodiments B26 to B28, wherein the concentration of the MNase in the supernatant is about 60 units/mL.

B30. The method of any one of embodiments B26 to B29, wherein the MNase is incubated with the solid support containing bound AAV particles.

B31. The method of any one of embodiments B1 to B30, wherein the AAV particles are eluted using a low pH buffer.

B32. The method of embodiment B31, further comprising a high pH buffer prior to the low pH buffer.

B33. The method of embodiment B31 or B32, wherein the low pH buffer is less than about pH 3.0.

B34. The method of embodiment B33, wherein the low pH buffer is about pH 1.5 to about pH 2.5.

B35. The method of embodiment B33, wherein the low pH buffer is about pH 1.5.

B36. The method of embodiment B33, wherein the low pH buffer is about pH 2.5.

B37. The method of any one of embodiments B31 to B36, wherein the low pH buffer is a citrate buffer, glycine buffer, or a phosphoric acid buffer.

B38. The method of embodiment B37, wherein the low pH buffer is a citrate buffer.

B39. The method of embodiment B37, wherein the low pH buffer is a phosphoric acid buffer.

B40. The method of any one of embodiments B14 to B39, wherein the affinity chromatography purification comprises two affinity chromatography purifications.

B41. The method of embodiment B40, wherein the method comprises an affinity chromatography purification followed by an anion-exchange chromatography.

B42. The method of any one of embodiments B31 to B41, further comprising neutralizing the pH of the low pH buffer.

B43. The method of embodiment B42, wherein neutralizing comprises adding Bis-Tris-Propane (BTP) or a Tris buffer.

B44. The method of any one of embodiments B31 to B43, further comprising about 5% to about 40% ethanol.

B45. The method of embodiment B44, comprising about 10% to about 30% ethanol.

B46. The method of embodiment B44 or B45, comprising about 15% to about 25% ethanol.

B47. The method of any one of embodiments B44 to B46, comprising about 20% ethanol.

B48. The method of any one of embodiments B1 to B47, wherein the purified AAV particles are substantially free of chromatin-associated DNA, when compared to non-MNase contacted purified AAV particles.

B49. The method of any one of embodiments B1 to B48, wherein the purified AAV particles are substantially free of host-cell DNA, when compared to non-MNase contacted purified AAV particles.

B50. The method of embodiment B49, wherein the host-cell DNA concentration is less than 2 ng/mL.

B51. The method of embodiment B49, wherein the host-cell DNA concentration is less than 1.5 ng/mL.

B52. The method of embodiment B49, wherein the host-cell DNA concentration is less than 1 ng/mL.

B53. The method of any one of embodiments B1 to B52, wherein the purified AAV particles are substantially free of host cell proteins, when compared to non-MNase contacted purified AAV particles.

B54. The method of embodiment B53, wherein the purified AAV particles are substantially free of a DNA binding protein, when compared to non-MNase contacted purified AAV particles.

B55. The method of embodiment B54, wherein the DNA binding protein comprises a histone.

B56. The method of any one of embodiments B1 to B55, wherein the purified AAV particles are substantially free of macroscopic and microscopic impurities.

B57. The method of any one of embodiments B1 to B56, wherein the viral titer is increased about 2 fold to about 100 fold.

B58. The method of any one of embodiments B1 to B56, wherein the viral titer is increased about 2 fold or greater.

B59. The method of any one of embodiments B1 to B56, wherein the viral titer is increased about 3 fold or greater.

B60. The method of any one of embodiments B1 to B56, wherein the viral titer is increased about 7 fold or greater.

B61. The method of any one of embodiments B1 to B56, wherein the viral titer is increased about 80 fold or greater.

B62. The method of any one of embodiments B1 to B61, wherein a viral titer ratio of the product AAV particle fraction to a post-product AAV particle fraction is increased, when compared to non-MNase contacted purified AAV particles.

B63. The method of embodiment B62, wherein the viral titer ratio is increased about 2 fold or greater.

B64. The method of embodiment B62, wherein the viral titer ratio is increased about 5 fold or greater.

B65. The method of embodiment B62, wherein the viral titer ratio is increased about 10 fold or greater.

B66. The method of embodiment B62, wherein the viral titer ratio is increased about 25 fold or greater.

B67. The method of any one of embodiments B1 to B66, wherein the purified AAV particles have a melting temperature (Tm) within less than about 10° C. of an aggregation temperature (Tagg), as measured by dynamic light scatter (DLS).

B68. The method of embodiment B67, wherein the purified AAV particles have a Tm within less than about 5° C. of an aggregation temperature (Tagg), as measured by dynamic light scatter (DLS).

B69. The method of embodiment B67, wherein the purified AAV particles have a melting temperature (Tm) within less than 2° C. of an aggregation temperature (Tagg), as measured by dynamic light scatter (DLS).

B70. The method of any one of embodiments B1 to B69, wherein the purified AAV particles comprise a full-to-empty capsid ratio of greater than about 50%.

B71. The method of embodiment B70, wherein the purified AAV particles comprise a full-to-empty capsid ratio of greater than about 60%.

B72. The method of embodiment B70, wherein the purified AAV particles comprise a full-to-empty capsid ratio of greater than about 70%.

B73. The method of any one of embodiments B1 to B72, wherein the purified AAV particles comprise a AAV particle post-product fraction with a reduced absorbance at 260 nm, when compared to non-MNase contacted purified AAV particles.

B74. The method of any one of embodiments B1 to B73, wherein the purified AAV particles comprise a AAV particle post-product fraction with a reduced absorbance at 280 nm, when compared to non-MNase contacted purified AAV particles.

In another set of embodiments, provided are:

C1. A composition of purified AAV particles, wherein the AAV particles have been purified by a purification method comprising a chromatin-DNA nuclease.

C2. The composition of embodiment C1, wherein the purification method comprises centrifugation, chromatography, filtration, or a combination thereof.

C3. The composition of embodiment C2, wherein centrifugation comprises density gradient centrifugation, ultracentrifugation, or a combination thereof.

C4. The composition of embodiment C2, wherein chromatography comprises affinity chromatography, ion exchange chromatography, size exclusion chromatography, hydrophobic interaction chromatography, or a combination thereof.

C5. The composition of embodiment C4, wherein the purification method further comprises incubating a supernatant comprising AAV particles with a solid support for a sufficient amount of time to bind the AAV particles.

C6. The composition of embodiment C5, wherein the purification method further comprises washing the solid support.

C7. The composition of embodiment C6, wherein the washing comprises a high pH buffer.

C8. The composition of embodiment C7, wherein the high pH buffer is greater than pH 9.0.

C9. The composition of embodiment C7, wherein the high pH buffer is between pH 9.0 and pH 11.

C10. The composition of embodiment C7, wherein the high pH buffer is about pH 9.5.

C11. The composition of embodiment C7, wherein the high pH buffer is about pH 10.2.

C12. The composition of embodiment C7, wherein the high pH buffer is about pH 10.3.

C13. The composition of embodiment C7, wherein the high pH buffer is about pH 10.4.

C14. The composition of any one of embodiments C4 to C13, wherein the purification method comprises one or more affinity chromatography purifications.

C15. The composition of embodiment C14, wherein the affinity chromatography comprises ion exchange chromatography.

C16. The composition of embodiment C15, wherein the ion exchange chromatography comprises anion exchange chromatography.

C17. The composition of any one of embodiments C5 to C16, wherein the supernatant is a clarified supernatant.

C18. The composition of any one of embodiments C1 to C17, wherein the purification method further comprises Benzonase®.

C19. The composition of any one of embodiments C5 to C18, wherein the purification method comprises elution with a low pH buffer.

C20. The composition of embodiment C19, wherein the purification method further comprises a high pH buffer prior to the low pH buffer.

C21. The composition of embodiment C19 or C20, wherein the low pH buffer is less than about pH 3.0.

C22. The composition of embodiment C21, wherein the low pH buffer is about pH 1.5 to about pH 2.5.

C23. The composition of embodiment C21, wherein the low pH buffer is about pH 1.5.

C24. The composition of embodiment C21, wherein the low pH buffer is about pH 2.5.

C25. The composition of any one of embodiments C19 to C24, wherein the low pH buffer is a citrate buffer, glycine buffer, or a phosphoric acid buffer.

C26. The composition of embodiment C25, wherein the low pH buffer is a citrate buffer.

C27. The composition of embodiment C25, wherein the low pH buffer is a phosphoric acid buffer.

C28. The composition of any one of embodiment C14 to C27, wherein the purification method comprises two affinity chromatography purifications.

C29. The composition of embodiment C28, wherein the purification method comprises an affinity chromatography purification followed by an anion-exchange chromatography.

C30. The composition of any one of embodiments C19 to C29, wherein the purification method further comprises neutralizing the pH of the low pH buffer.

C31. The composition of embodiment C30, wherein neutralizing comprises adding Bis-Tris-Propane (BTP) or a Tris buffer.

C32. The composition of any one of embodiments C19 to C31, wherein the purification method further comprises elution with about 5% to about 40% ethanol.

C33. The composition of embodiment C32, wherein the purification method comprises about 10% to about 30% ethanol.

C34. The composition of embodiment C32 or C33, wherein the purification method comprises about 15% to about 25% ethanol.

C35. The composition of any one of embodiments C32 to C34, wherein the purification method comprises about 20% ethanol.

C36. The composition of any one of embodiments C1 to C35, wherein the composition is substantially free of an impurity, when compared to a composition purified by a method not comprising a chromatin-DNA nuclease.

C37. The composition of any one of embodiments C1 to C36, wherein the composition is substantially free of chromatin-associated DNA, when compared to a composition purified by a method not comprising a chromatin-DNA nuclease.

C38. The composition of any one of embodiments C1 to C37, wherein the composition is substantially free of host-cell DNA, when compared to a composition purified by a method not comprising a chromatin-DNA nuclease.

C39. The composition of embodiment C38, wherein the host-cell DNA concentration is less than 2 ng/mL.

C40. The composition of embodiment C38, wherein the host-cell DNA concentration is less than 1.5 ng/mL.

C41. The composition of embodiment C38, wherein the host-cell DNA concentration is less than 1 ng/mL.

C42. The composition of any one of embodiments C1 to C41, wherein the composition is substantially free of host cell proteins, when compared to a composition not contacted with a chromatin-DNA nuclease.

C43. The composition of any one of embodiments C1 to C42, wherein the composition is substantially free of a DNA binding protein, when compared to a composition not contacted with a chromatin-DNA nuclease.

C44. The composition of embodiment C43, wherein the DNA binding protein comprises a histone.

C45. The composition of any one of embodiments C1 to C44, wherein the composition is substantially free of macroscopic and microscopic impurities.

C46. The composition of any one of embodiments C1 to C45, wherein the composition comprises a reduced post-product AAV particle fraction peak as measured by anion exchange chromatogram, when compared to a composition not contacted with a chromatin-DNA nuclease.

C47. The composition of any one of embodiments C1 to C45, wherein the composition comprises an increased viral titer, when compared to a composition not contacted with a chromatin-DNA nuclease.

C48. The composition of embodiment C47, wherein the viral titer comprises a physical viral titer, a functional viral titer, or both.

C49. The composition of embodiment C47, wherein the viral titer comprises a physical titer.

C50. The composition of embodiment C47, wherein the viral titer comprises a functional titer.

C51. The composition of any one of embodiments C47 to C50, wherein the viral titer is increased about 2 fold to about 100 fold.

C52. The composition of any one of embodiments C47 to C50, wherein the viral titer is increased about 2 fold or greater.

C53. The composition of any one of embodiments C47 to C50, wherein the viral titer is increased about 3 fold or greater.

C54. The composition of any one of embodiments C47 to C50, wherein the viral titer is increased about 7 fold or greater.

C55. The composition of any one of embodiments C47 to C50, wherein the viral titer is increased about 80 fold or greater.

C56. The composition of any one of embodiments C1 to C55, wherein the composition comprises an increased viral titer ratio of a product AAV particle fraction to a post-product AAV particle fraction, when compared to a composition not contacted with a chromatin-DNA nuclease.

C57. The composition of embodiment C56, wherein the viral titer ratio is increased about 2 fold or greater.

C58. The composition of embodiment C56, wherein the viral titer ratio is increased about 5 fold or greater.

C59. The composition of embodiment C56, wherein the viral titer ratio is increased about 10 fold or greater.

C60. The composition of embodiment C56, wherein the viral titer ratio is increased about 25 fold or greater.

C61. The composition of any one of embodiments C1 to C60, wherein the purified AAV particles have a melting temperature (Tm) within less than about 10° C. of an aggregation temperature (Tagg), as measured by dynamic light scatter (DLS).

C62. The composition of embodiment C61, wherein the purified AAV particles have a Tm within less than about 5° C. of an aggregation temperature (Tagg), as measured by dynamic light scatter (DLS).

C63. The composition of embodiment C61, wherein the purified AAV particles have a melting temperature (Tm) within less than 2° C. of an aggregation temperature (Tagg), as measured by dynamic light scatter (DLS).

C64. The composition of any one of embodiments C1 to C63, wherein the composition comprises a full-to-empty capsid ratio of greater than about 50%.

C65. The composition of embodiment C64, wherein the composition comprises a full-to-empty capsid ratio of greater than about 60%.

C66. The composition of embodiment C64, wherein the composition comprises a full-to-empty capsid ratio of greater than about 70%.

C67. The composition of any one of embodiments C1 to C66, wherein the purified AAV particles comprise a AAV particle post-product fraction with a reduced absorbance at 260 nm, when compared to a composition not contacted with a chromatin-DNA nuclease.

C68. The composition of any one of embodiments C1 to C67, wherein the purified AAV particles comprise a AAV particle post-product fraction with a reduced absorbance at 280 nm, when compared to a composition not contacted with a chromatin-DNA nuclease.

C69. The composition of any one of embodiments C1 to C68, wherein the chromatin-DNA nuclease is MNase.

In another set of embodiments, provided are:

D1. A composition for use in producing an AAV particle that is substantially free of chromatin-associated DNA, the composition comprising:

(a) a supernatant comprising AAV particles; and

(b) a chromatin-DNA nuclease.

D2. The composition of embodiment D1, further comprising Benzonase®.

D3. The composition of embodiment D1 or D2, wherein the chromatin-DNA nuclease is micrococcal nuclease (MNase).

D4. The composition of embodiment D3, wherein the concentration of the MNase in the supernatant is greater than 2.5 units/mL.

D5. The composition of embodiment D4, wherein the concentration of the MNase in the supernatant is greater than 10 units/mL.

D6. The composition of embodiment D4 or D5, wherein the concentration of the MNase in the supernatant is about 30 units/mL to about 100 units/mL.

D7. The composition of any one of embodiments D4 to D6, wherein the concentration of the MNase in the supernatant is about 60 units/mL.

D8. The composition of embodiment D3, wherein the MNase is present in a sufficient amount to digest chromatin associated with an AAV particle.

D9. A composition, comprising:

(a) a supernatant comprising AAV particles; and

(b) a chromatin-DNA nuclease.

D10. The composition of embodiment D9, further comprising Benzonase®.

D11. The composition of embodiment D9 or D10, wherein the chromatin-DNA nuclease is micrococcal nuclease (MNase).

D12. The composition of embodiment D11, wherein the concentration of the MNase is greater than 2.5 units/mL.

D13. The composition of embodiment D11, wherein the concentration of the MNase is greater than 10 units/mL.

D14. The composition of embodiment D11, wherein the concentration of the MNase is about 30 units/mL to about 100 units/mL.

D15. The composition of any one of embodiments D11 to D13, wherein the concentration of the MNase in the supernatant is about 60 units/mL.

D16. The composition of embodiment D11, wherein the MNase is present in a sufficient amount to reduce AAV particle impurities.

D17. The composition of embodiment D16, wherein the AAV particle impurities comprise one or more of a host-cell DNA, a host-cell protein, a chromatin-associated DNA, and a DNA binding protein.

D18. The composition of embodiment D16 or D17, wherein the AAV particle impurities comprise macroscopic and microscopic impurities.

D19. The composition of embodiment D17, wherein the DNA binding protein comprises a histone.

D20. The composition of embodiment D11, wherein the MNase is present in an amount sufficient to increase a viral titer of AAV particles.

D21. The composition of embodiment D20, wherein the viral titer comprises a physical viral titer, a functional viral titer, or both.

D22. The composition of embodiment D20, wherein the viral titer comprises a physical titer.

D23. The composition of embodiment D20, wherein the viral titer comprises a functional titer.

D24. The composition of any one of embodiments D20 to D23, wherein the viral titer is increased about 2 fold to about 100 fold.

D25. The composition of any one of embodiments D20 to D23, wherein the viral titer is increased about 2 fold or greater.

D26. The composition of any one of embodiments D20 to D23, wherein the viral titer is increased about 3 fold or greater.

D27. The composition of any one of embodiments D20 to D23, wherein the viral titer is increased about 7 fold or greater.

D28. The composition of any one of embodiments D20 to D23, wherein the viral titer is increased about 80 fold or greater.

D29. The composition of any one of embodiments D20 to D28, wherein the MNase is present in a sufficient amount to increase a viral titer ratio of a product AAV particle fraction to a post-product AAV particle fraction.

D30. The composition of embodiment D29, wherein the viral titer ratio is increased about 2 fold or greater.

D31. The composition of embodiment D29, wherein the viral titer ratio is increased about 5 fold or greater.

D32. The composition of embodiment D29, wherein the viral titer ratio is increased about 10 fold or greater.

D33. The composition of embodiment D29, wherein the viral titer ratio is increased about 25 fold or greater.

D34. The composition of embodiment D11, wherein the MNase is present in an amount sufficient to increase a full-to-empty capsid ratio.

D35. The composition of embodiment D34, wherein the full-to-empty capsid ratio is greater than about 50%.

D36. The composition of embodiment D34, wherein the full-to-empty capsid ratio is greater than about 60%.

D37. The composition of embodiment D34, wherein the full-to-empty capsid ratio is greater than about 70%.

D38. The composition of embodiment D11, wherein the MNase is present in an amount sufficient to decrease a AAV particle post-product fraction, as measured by absorbance at 260 nm.

D39. The composition of embodiment D11, wherein the MNase is present in an amount sufficient to decrease a AAV particle post-product fraction, as measured by absorbance at 280 nm.

D40. The composition of embodiment D11, wherein the MNase is present in an amount sufficient to produce purified AAV particles comprising a melting temperature (Tm) within less than about 10° C. of an aggregation temperature (Tagg), as measured by dynamic light scatter (DLS).

D41. The composition of embodiment D11, wherein the MNase is present in an amount sufficient to produce purified AAV particles comprising a melting temperature (Tm) within less than about 5° C. of an aggregation temperature (Tagg), as measured by dynamic light scatter (DLS).

D42. The composition of embodiment D11, wherein the MNase is present in an amount sufficient to produce purified AAV particles comprising a melting temperature (Tm) within less than about 2° C. of an aggregation temperature (Tagg), as measured by dynamic light scatter (DLS).

In another set of embodiments, provided are:

E1. A kit comprising,

(a) Benzonase®; and

(b) a chromatin-DNA nuclease.

E2. The kit of embodiment E1, wherein the chromatin-DNA nuclease is micrococcal nuclease (MNase).

E3. The kit of embodiment E2, wherein the concentration of the MNase is greater than 2.5 units/mL.

E4. The kit of embodiment E2, wherein the concentration of the MNase is greater than 10 units/mL.

E5. The kit of embodiment E2, wherein the concentration of the MNase is about 30 units/mL to about 100 units/mL.

E6. The kit of embodiment E2, wherein the concentration of the MNase is about 60 units/mL.

E7. The kit of embodiment E2, wherein the MNase is present in a sufficient amount to digest chromatin associated with an AAV particle.

E8. The kit of embodiment E2 or E7, wherein the MNase is present in a sufficient amount to reduce AAV particle impurities.

E9. The kit of embodiment E8, wherein the AAV particle impurities comprise one or more of a host-cell DNA, a host-cell protein, a chromatin-associated DNA, and a DNA binding protein.

E10. The kit of embodiment E8 or E9, wherein the AAV particle impurities comprise macroscopic and microscopic impurities.

E11. The kit of embodiment E9, wherein the DNA binding protein comprises a histone.

E12. The kit of embodiment E2, wherein the MNase is present in an amount sufficient to increase a viral titer of AAV particles.

E13. The kit of embodiment E12, wherein the viral titer comprises a physical viral titer, a functional viral titer, or both.

E14. The kit of embodiment E12, wherein the viral titer comprises a physical titer.

E15. The kit of embodiment E12, wherein the viral titer comprises a functional titer.

E16. The kit of any one of embodiments E12 to E15, wherein the viral titer is increased about 2 fold to about 100 fold.

E17. The kit of any one of embodiments E12 to E15, wherein the viral titer is increased about 2 fold or greater.

E18. The kit of any one of embodiments E12 to E15, wherein the viral titer is increased about 3 fold or greater.

E19. The kit of any one of embodiments E12 to E15, wherein the viral titer is increased about 7 fold or greater.

E20. The kit of any one of embodiments E12 to E15, wherein the viral titer is increased about 80 fold or greater.

E21. The kit of any one of embodiments E12 to E20, wherein the MNase is present in a sufficient amount to increase a viral titer ratio of a product AAV particle fraction to a post-product AAV particle fraction.

E22. The kit of embodiment E21, wherein the viral titer ratio is increased about 2 fold or greater.

E23. The kit of embodiment E21, wherein the viral titer ratio is increased about 5 fold or greater.

E24. The kit of embodiment E21, wherein the viral titer ratio is increased about 10 fold or greater.

E25. The kit of embodiment E21, wherein the viral titer ratio is increased about 25 fold or greater.

E26. The kit of embodiment E2, wherein the MNase is present in an amount sufficient to increase a full-to-empty capsid ratio.

E27. The kit of embodiment E26, wherein the full-to-empty capsid ratio is greater than about 50%.

E28. The kit of embodiment E26, wherein the full-to-empty capsid ratio is greater than about 60%.

E29. The kit of embodiment E26, wherein the full-to-empty capsid ratio is greater than about 70%.

E30. The kit of embodiment E2, wherein the MNase is present in an amount sufficient to decrease a AAV particle post-product fraction, as measured by absorbance at 260 nm.

E31. The kit of embodiment E2, wherein the MNase is present in an amount sufficient to decrease a AAV particle post-product fraction, as measured by absorbance at 280 nm.

E32. The kit of embodiment E2, wherein the MNase is present in an amount sufficient to produce purified AAV particles comprising a melting temperature (Tm) within less than about 10° C. of an aggregation temperature (Tagg), as measured by dynamic light scatter (DLS).

E33. The kit of embodiment E2, wherein the MNase is present in an amount sufficient to produce purified AAV particles comprising a melting temperature (Tm) within less than about 5° C. of an aggregation temperature (Tagg), as measured by dynamic light scatter (DLS).

E34. The kit of embodiment E2, wherein the MNase is present in an amount sufficient to produce purified AAV particles comprising a melting temperature (Tm) within less than about 2° C. of an aggregation temperature (Tagg), as measured by dynamic light scatter (DLS).

In another set of embodiments, provided are:

F1. A composition comprising a means for decreasing an impurity in purified AAV particles.

F2. The composition of embodiment F1, wherein the impurity is selected from the group consisting of a host-cell DNA, a host-cell protein, a chromatin-associated DNA, and a DNA binding protein.

F3. The composition of embodiment F2, wherein the DNA binding protein comprises a histone.

F4. The composition of embodiment F1, wherein the impurity is a macroscopic impurity, a microscopic impurity, or both.

F5. A composition comprising a means for increasing a viral titer of AAV particles.

F6. The composition of embodiment F5, wherein the viral titer comprises a physical viral titer, a functional viral titer, or both.

F7. The composition of embodiment F5, wherein the viral titer comprises a physical viral titer.

F8. The composition of embodiment F5, wherein the viral titer comprises a functional viral titer.

F9. The composition of any one of embodiments F5 to F8, wherein the viral titer is increased about 2 fold to about 100 fold.

F10. The composition of any one of embodiments F5 to F8, wherein the viral titer is increased about 2 fold or greater.

F11. The composition of any one of embodiments F5 to F8, wherein the viral titer is increased about 3 fold or greater.

F12. The composition of any one of embodiments F5 to F8, wherein the viral titer is increased about 7 fold or greater.

F13. The composition of any one of embodiments F5 to F8, wherein the viral titer is increased about 80 fold or greater.

F14. A composition comprising a means for increasing a viral titer ratio of a product AAV particle fraction to a post-product AAV particle fraction.

F15. The composition of embodiment F14, wherein the viral titer ratio is increased about 2 fold or greater.

F16. The composition of embodiment F14, wherein the viral titer ratio is increased about 5 fold or greater.

F17. The composition of embodiment F14, wherein the viral titer ratio is increased about 10 fold or greater.

F18. The composition of embodiment F14, wherein the viral titer ratio is increased about 25 fold or greater.

F19. A composition comprising a means for increasing the full-to-empty capsid ratio of AAV particles.

F20. The composition of embodiment F19, wherein the full-to-empty capsid ratio is greater than about 50%.

F21. The composition of embodiment F19, wherein the full-to-empty capsid ratio is greater than about 60%.

F22. The composition of embodiment F19, wherein the full-to-empty capsid ratio is greater than about 70%.

F23. A composition comprising a means to decrease a AAV particle post-product fraction, as measured by absorbance at 260 nm.

F24. A composition comprising a means to decrease a AAV particle post-product fraction, as measured by absorbance at 280 nm.

F25. A composition comprising a first means to remove a DNA binding protein extra-virally complexed to an AAV particle and a second means to remove residual host production cell nucleic acids and/or proteins.

F26. A method of purifying an AAV particle comprising (i) a step for removing a DNA binding protein extra-virally complexed to an AAV particle.

F27. The method of embodiment F26, further comprising (ii) a second step for removing residual host production cell nucleic acids and/or proteins.

F28. The method of embodiment F26 or F27, further comprising (iii) a third step for increasing a viral titer.

In another set of embodiments, provided are:

G1. A system comprising a means for making and obtaining a purified AAV particle substantially free of an impurity.

G2. The system of embodiment G1, wherein the impurity is selected from the group consisting of a host-cell DNA, a host-cell protein, a chromatin-associated DNA, and a DNA binding protein.

G3. The system of embodiment G2, wherein the DNA binding protein comprises a histone.

G4. The system of embodiment G1, wherein the impurity is a macroscopic impurity, a microscopic impurity, or both.

G5. A system comprising a means for making and obtaining AAV particles with an increased viral titer.

G6. The system of embodiments G5, wherein the viral titer comprises a physical viral titer, a functional viral titer, or both.

G7. The system of embodiments G5, wherein the viral titer comprises a physical viral titer.

G8. The system of embodiments G5, wherein the viral titer comprises a functional viral titer.

G9. The system of any one of embodiments G5 to G8, wherein the viral titer is increased about 2 fold to about 100 fold.

G10. The system of any one of embodiments G5 to G8, wherein the viral titer is increased about 2 fold or greater.

G11. The system of any one of embodiments G5 to G8, wherein the viral titer is increased about 3 fold or greater.

G12. The system of any one of embodiments G5 to G8, wherein the viral titer is increased about 7 fold or greater.

G13. The system of any one of embodiments G5 to G8, wherein the viral titer is increased about 80 fold or greater.

G14. A system comprising a means for increasing a viral titer ratio of a product AAV particle fraction to a post-product AAV particle fraction.

G15. The system of embodiment G14, wherein the viral titer ratio is increased about 2 fold or greater.

G16. The system of embodiment G14, wherein the viral titer ratio is increased about 5 fold or greater.

G17. The system of embodiment G14, wherein the viral titer ratio is increased about 10 fold or greater.

G18. The system of embodiment G14, wherein viral titer ratio is increased about 25 fold or greater.

G19. A system comprising a means for increasing the full-to-empty capsid ratio of AAV particles.

G20. The system of embodiment G19, wherein the full-to-empty capsid ratio is greater than about 50%.

G21. The system of embodiment G19, wherein the full-to-empty capsid ratio is greater than about 60%.

G22. The system of embodiment G19, wherein the full-to-empty capsid ratio is greater than about 70%.

G23. A system comprising a means to decrease a AAV particle post-product fraction, as measured by absorbance at 260 nm.

G24. A system comprising a means to decrease a AAV particle post-product fraction, as measured by absorbance at 280 nm.

G25. A system comprising a first means to remove DNA binding proteins extra-virally complexed to AAV particles and a second means to remove residual nucleic acids from a host production cell.

### EXAMPLES

The following examples of the application are to further illustrate the nature of the application. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Example I

### Extra-Viral DNA Binding Protein Associated with AAV Particles Identified as an Impurity Post-Purification

This example establishes that following down-stream purification of AAV particles, residual DNA binding protein/chromatin complexes associating with AAV particles can be detected and released by the addition of a chromatin-DNA nuclease, micrococcal nuclease (MNase).

Briefly, clarified (0.2 μm filtered) supernatant of suspension culture containing AAV8 particles was subjected to affinity exchange chromatography using the commercially available POROS CaptureSelect AAVX affinity resin (Thermo) coupled to an anion-exchange polish step using the CIMmultus QA-monolithic column (BIA Separations). Clarified supernatants from AAV particle production cultures loaded over the POROS column at a constant flow rate of 5.5 mL/min. Virus containing supernatant was bound to the column at normal pH (7.5). To reduce viscosity and increased pre-column pressure build-up over time, 5 U/mL of Benzonase® was added during purification. Subsequently, the virus was eluted using a low pH (2.5) citrate-buffer and immediately neutralized with BTP pH 10.2.

The second phase of viral purification involved an anion-exchange polish using a high ionic strength buffer at pH 10, a pH higher than the predicted pKa for the virus. At this pH, AAV particles will bind tightly to the monolith, reduce viral aggregation and further eliminate residual host cell proteins or DNA. Viruses were eluted using an increasing salt gradient and based on differences in charge between DNA containing “full-capsids” vs “empty capsids”, the desired viral population was separated as the product peak. However, multiple species of ‘post-product peaks’ were routinely observed during anion exchange polish.

Given that the elution profiles of these post-product peaks were heterogeneous and required higher ionic strength, relative to the product peak, it was suspected that these fractions of viruses could still be associated with highly charge proteins. Based on this observation, interaction between AAV particles and DNA containing complexes that are resistant to Benzonase® digestion was investigated. Benzonase® is relatively ineffective at digesting nucleosomal DNA, so investigation into whether the higher ionic strength required to elute these virus particles was a direct result of residual DNA binding protein/chromatin complexes associating with AAV particles was performed.

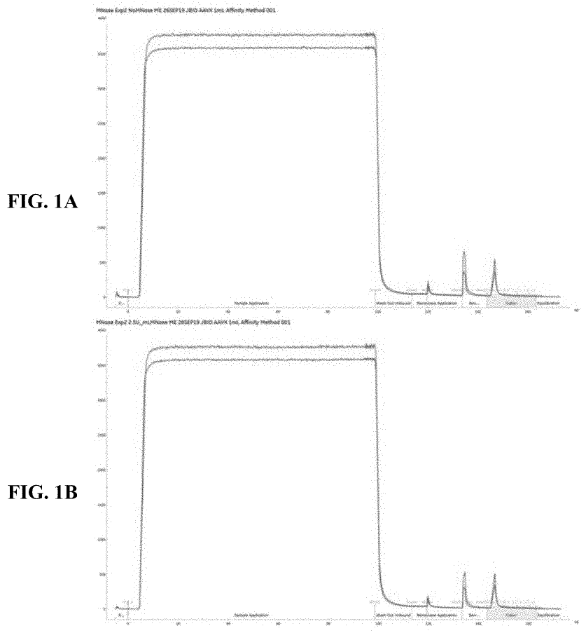

To test the hypothesis that chromatin structures are associating extra-virally with AAV particles, capsids were subjected to increased nucleic acid enzymatic digestion using MNase either alone or in combination with Benzonase®. Benzonase® was added with or without MNase, directly to the AAVX-containing bound virus. This step was held for 30 minutes to digest DNA and chromatin-associated DNA. Increasing amounts of MNase was added either in combination or alone to the ‘on-column’ Benzonase® step during affinity chromatography step. The results indicated the shape of the chromatogram was the same for samples treated without MNase (FIG. 1A) or with MNase (FIG. 1B), and that MNase did not affect the yield of virus.

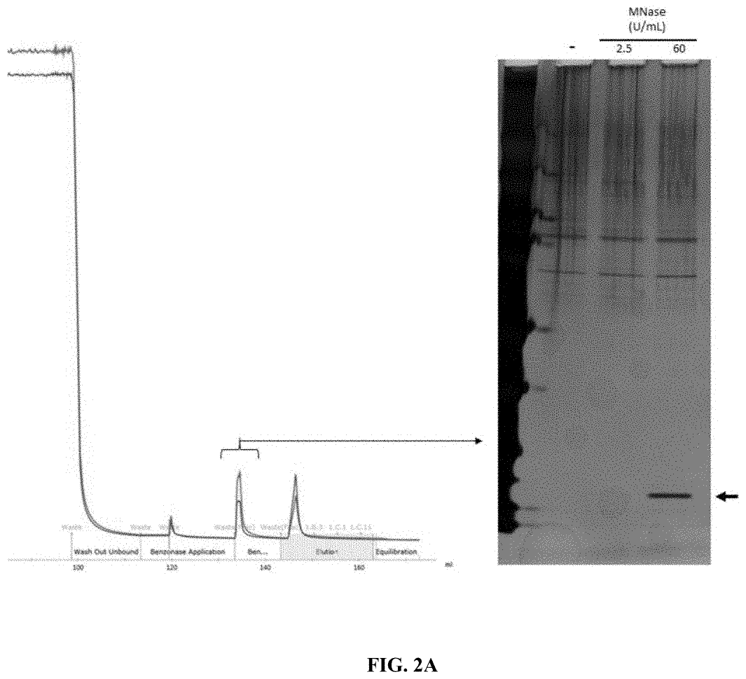

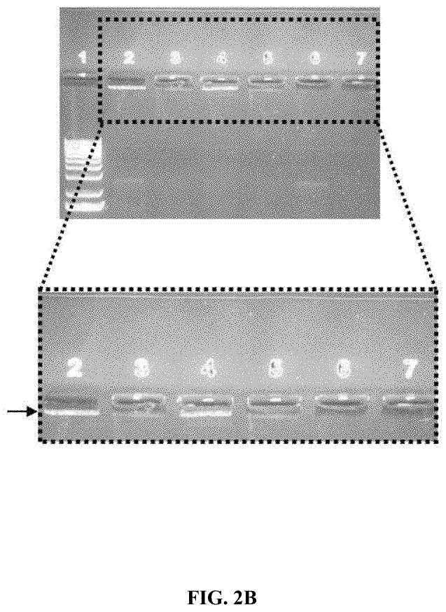

The post-column enzymatic wash-out was collected and subjected to sodium dodecyl sulfate-polyacrylamide gel electrophoresis (SDS-PAGE) followed by silver staining for the presence of a DNA binding protein. Silverstain analysis showed the presence of a single protein band at ˜10 kDa, only identifiable in optimal (60 U/mL) MNase-treated samples (FIG. 2A, third lane). Sub-optimal (2.5 U/mL) concentrations of MNase with or without Benzonase® alone was inefficient at releasing extra-virally associated DNA binding proteins (FIG. 2A, second lane). This indicated that 60 U/mL of MNase was able to release the chromatin-bound DNA binding proteins.

In addition, the post-product peak fractions were analyzed by electrophoresis to visualize any chromatin that may be present in the sample. The results indicated that treatment with MNase during affinity capture chromatography was sufficient to remove chromatin from the post-product peak fraction (FIG. 2B). Specifically, the following samples were analyzed: undiluted sample (Lane 2) and a 1:10 diluted sample (Lane 3) from rAAV8 particles produced in suspension Expi293F™ cells using the ExpiFectamine™ 293 Transfection Kit; undiluted sample (Lane 4) and a 1:10 diluted sample (Lane 5) rAAV8 particles produced in suspension Expi293F™ cells without the transfection kit; and undiluted sample (Lane 6) and a 1:10 diluted sample (Lane 7) from rAAV8 particles produced in suspension Expi293F™ cells without the transfection kit and digested with 60 U/mL MNase at 25° C. for 30 minutes. The results demonstrated that there was a visible high-molecular weight DNA in post-product peaks for the non-MNase treated samples (e.g., Lanes 2 and 4), which was indicative of chromatin since chromatin is too large to penetrate the gel. In contrast, high-molecular weight DNA was absent in MNase treated samples (e.g., Lane 6).

These results indicated that AAV particles contain residual DNA binding proteins/chromatin complexes that associate with AAV particles, and that the complexes can be disrupted by including MNase to the purification process.

### Example II

### MNase Addition Improves Anion-Exchange Elution Profiles

This example establishes that the addition of a chromatin-DNA nuclease, micrococcal nuclease (MNase), significantly improves the quality of AAV particles recovered during down-stream purification.

Extra-viral, chromatin-associated AAV particles are an undesirable product, and it can cause visible precipitation of purified product, which can be problematic for any formulation studies. Moreover, increased chromatin/DNA binding protein are undesirable contaminants, both of which can increase host-cell protein/DNA contamination. To overcome the problems of chromatin-associated AAV particles, the present studies demonstrated that the chromatin-DNA nuclease, MNase, can be added during purification and results in an improved purification of AAV particles.

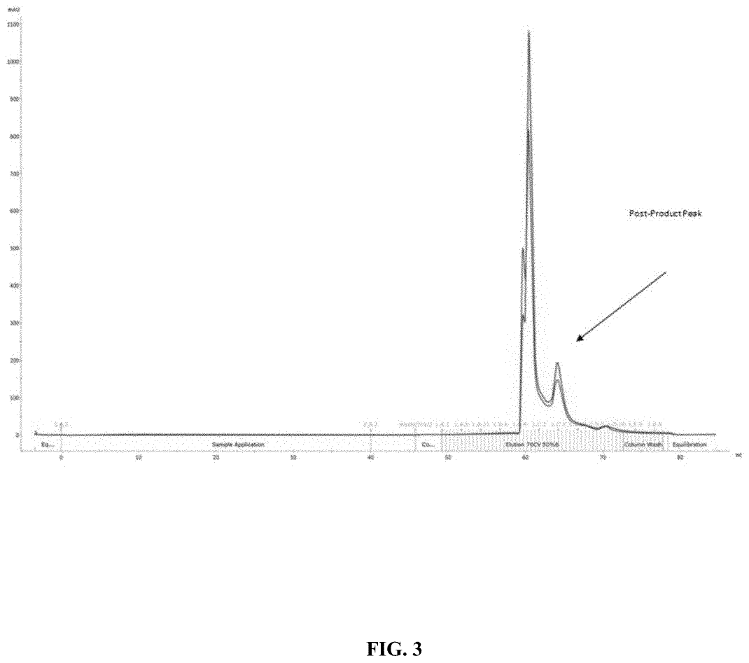

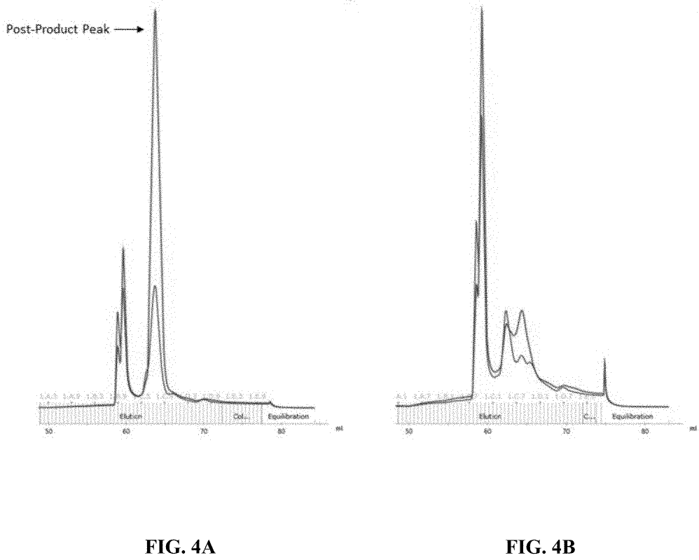

Production of AAV particles using adherent cells was observed to result in minimal cell death, and production of an insignificant post-product peak by anion exchange chromatogram (FIG. 3). In contrast, viability in suspension cell culture is reduced (approaching 50-70%), as compared to adherent cell cultures, and resulted in a predominant post-product peak (FIG. 4A). However, the addition of MNase during the affinity chromatography step reduced the height of post-product peak during anion-exchange chromatography, which indicated an improvement in the purity of the product (FIG. 4B). Specifically, 60 U/mL of MNase treatment reduced both the large 260 nm (DNA/RNA) absorbance peak, and the 280 nm (protein) absorbance peak of the post-product peak (FIG. 4B). A direct comparison of the post-product peak heights for DNA/RNA (260 nm) and protein (280 nm) by overlaying the affinity exchange chromatogram of rAAV8 particles produced from suspension culture with or without MNase treatment provided further evidence that MNase treatment for chromatin digestion enhances purification of AAV particles, and significantly reduced, to undetectable levels, residual host cell protein contamination (FIG. 5A and FIG. 5B, respectively). In particular, the reduction observed with the nucleic acid spectrum (260 nm), post-product peak height was quite significant, estimated to be over 90% reduction in peak height and area (FIG. 5A). In addition, a reduction in the protein spectrum (280 nm) post-product peak was also observed (FIG. 5B).

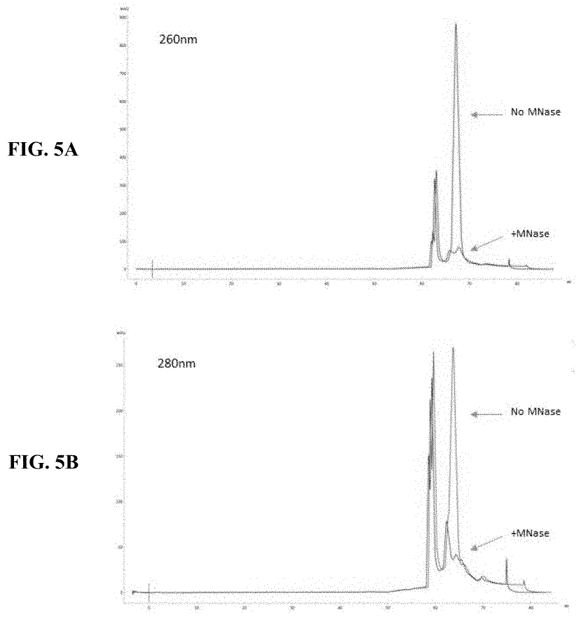

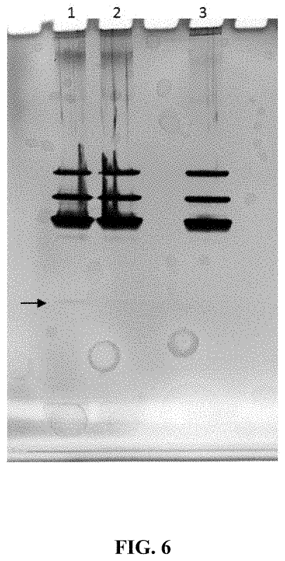

Moreover, silver stain analysis of post-product peaks after anion exchange chromatography revealed protein impurities present in the non-MNase digested samples, including a band at ˜10 kDa indicating the presence of DNA binding proteins, which was absent in MNase digested samples (FIG. 6A). Comparison of samples separated by gel electrophoresis and stained using silver staining revealed a faint band that corresponded with a molecular weight of ˜10 kDA, which is near the expected size of histones. Specifically, rAAV8 particles produced in suspension Expi293F™ cells (Thermo Fisher Scientific) using the ExpiFectamine™ 293 Transfection Kit Enhancer (Thermo Fisher Scientific) (Lane 1), rAAV8 particles produced in suspension Expi293F™ cells without Enhancer (Lane 2), and rAAV8 particles produced in suspension Expi293F™ cells without Enhancer and digested with 60 U/mL MNase at 25° C. for 30 minutes (Lane 3) were analyzed. The arrow indicates that the 10 kDA band was present in the non-MNase digested samples, but absent from the MNase digested sample (FIG. 6A). Additional evidence that large amounts of chromatin-associated DNA are normally associated extra-virally with AAV particles during purification was provided by the observation that increased salt was required to elute AAV particles in the post-product peak fraction, and the fact that DNA binding proteins, such as histones, are highly positively charged (behave like an anion exchanger).

To determine whether the elution buffer effected the anion-exchange elution profiles three different elution buffers were used: phosphate buffer, citrate buffer, and a high pH buffer. Measurement of the area for the product peal (“Peak A”), compared to the post-product peaks (any additional peaks detected beyond Peak A), for both 260 nm and 280 nm revealed that MNase treatment improved the amount of DNA/RNA in the product peak for all buffers (Table 1).

TABLE 1
Anion-exchange elution profiles for
DNA/RNA (260 nm) and protein (280 nm)

| | | 260 nm | | 280 nm | |
|---|---|---|---|---|---|
| | | No MNase | MNase | No MNase | MNase |
| Phosphate | Peak A | 43.55 | 49.33 | 42.13 | 54.23 |
| | Peak B | 47.75 | 37.91 | 50.07 | 38.78 |
| | Peak C | 8.71 | 7.94 | 7.80 | 6.99 |
| | Peak D | | 4.83 | | |
| Citrate | Peak A | 57.87 | 75.93 | 51.11 | 59.41 |
| | Peak B | 30.05 | 13.08 | 37.11 | 27.48 |
| | Peak C | 8.50 | 8.39 | 6.16 | 13.11 |
| | Peak D | 3.58 | 2.61 | 5.62 | |
| High pH | Peak A | 42.28 | 54.98 | 86.73 | 60.62 |
| | Peak B | 42.46 | 32.88 | 7.86 | 5.4133.51 |
| | Peak C | 4.48 | 7.31 | | 5.87 |
| | Peak D | 7.06 | 4.83 | | |
| | Peak E | 1.83 | | | |
| | Peak F | 1.79 | | | |

Taken together, these findings indicated that large amounts of chromatin-associated DNA are normally associated extra-virally with AAV particles during purification and that MNase treatment can enhance purification of AAV particles.

### Example III

### Increasing MNase Reduced Post-Product Peaks in Anion Exchange Chromatography

This example evidences that increasing MNase addition during affinity purification reduced chromatin-associated AAV particles in anion exchange chromatography polish step.

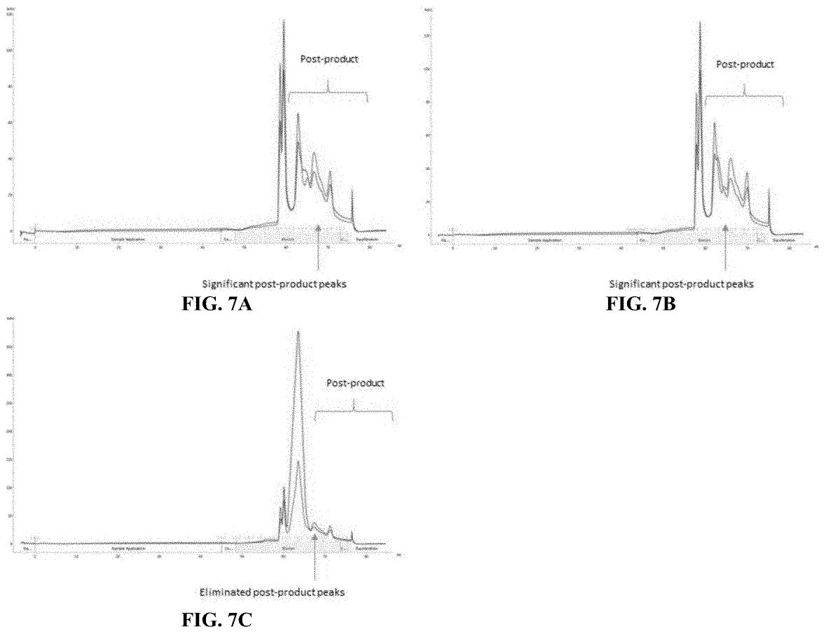

To determine an optimal range of MNase, samples were untreated (FIG. 7A), or treated with different amounts of MNase, e.g., 2.5 U/mL (FIG. 7B) or 60 U/mL (FIG. 7C). The affinity chromatography results indicated that higher amounts of MNase reduced the post-product peak (FIG. 7C), as compared to no MNase (FIG. 7A) or low amount of MNase (FIG. 7B).

These results demonstrated that increasing MNase addition during affinity purification reduced chromatin-associated AAV particles in anion exchange chromatography polish step, and evidenced that 60 U/mL of MNase was an effective concentration for reducing chromatin-associated AAV particles.

### Example IV

### MNase Digestion Prevented Aggregation and Reduced Precipitation of AAV Particles

This example establishes that MNase digestion prevented aggregation and precipitation of AAV particles.

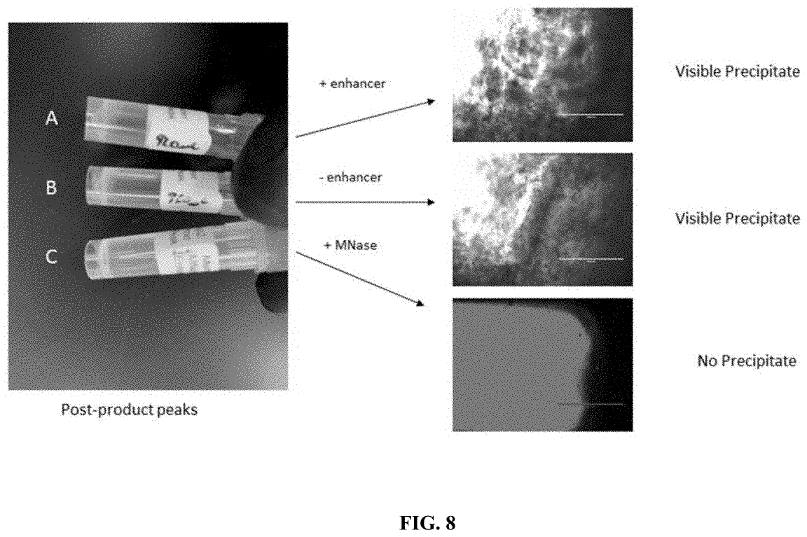

Macroscopic examination of the post-product peak fractions showed that non-MNase treated rAAV8 particles produced in suspension Expi293F™ cells (Thermo Fisher Scientific) using the ExpiFectamine™ 293 Transfection Kit Enhancer (Thermo Fisher Scientific), or non-MNase treated rAAV8 particles produced in suspension Expi293F™ cells without Enhancer had a visible precipitate under macroscopic examination (FIG. 8). In contrast, rAAV8 particles produced in suspension Expi293F™ cells without Enhancer and digested with 60 U/mL MNase at 25° C. for 30 minutes) had no visible precipitate (FIG. 8).

This data demonstrated that MNase digestion prevented aggregation and precipitation of AAV particles.

### Example VI

### MNase Treatment Increased Titers of AAV Particles

This example establishes that MNase treatment increased titers of AAV particles when it was added to different purification protocols.

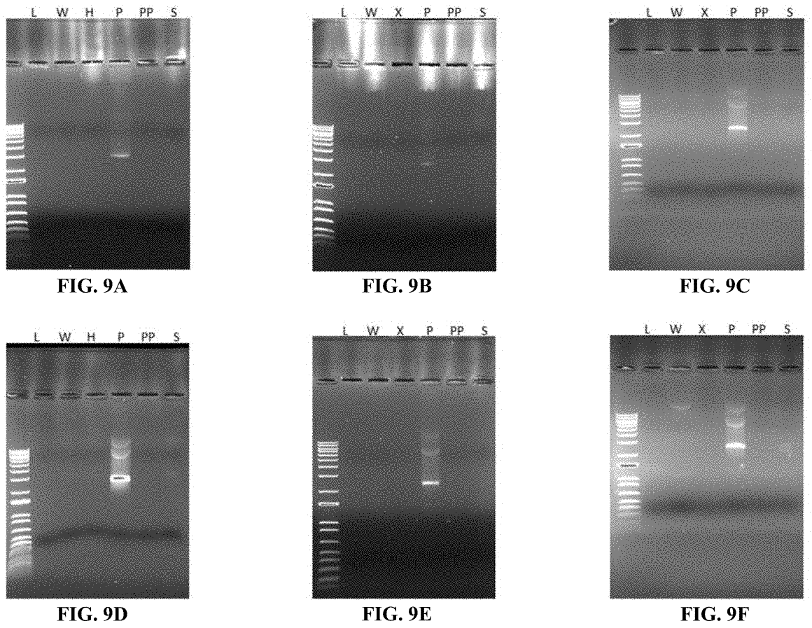

To determine whether MNase treatment was compatible with different elution methods, three different types of elution were performed using high/low pH elution, citrate elution, and low pH elution. A qualitative comparison between the DNA yields with or without MNase treatment was conducted using SYBR Gold staining after gel electrophoresis (FIG. 9A-FIG. 9F). The results indicated that samples treated with MNase had increased amounts of DNA in the product peak, as compared to non-MNase treated samples, using three different types of elution: High/Low pH elution, citrate elution, and low pH elution (FIG. 9A-FIG. 9F).

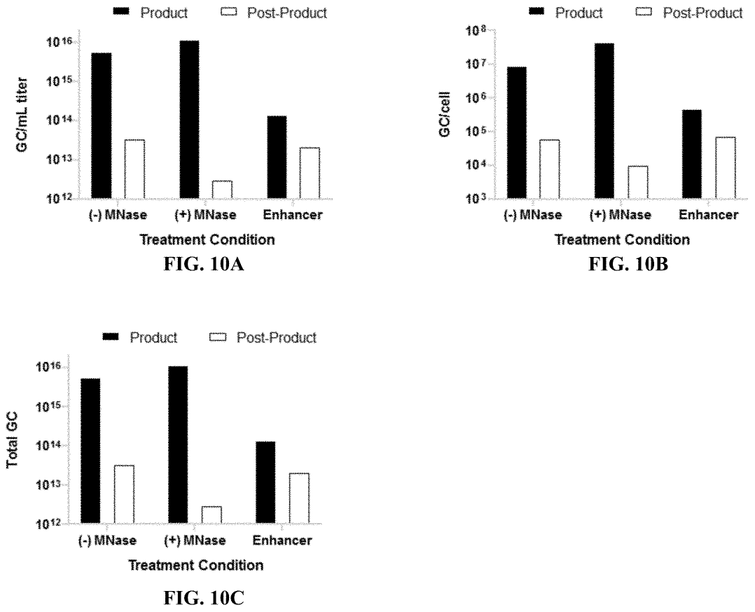

In addition, quantification of the genome copies per mL (GC/mL) titers (FIG. 10), genome copies per cell (GC/cell) (FIG. 10B), and total genome copies (FIG. 10C) revealed that MNase treatment increased viral titers of the desired product and reduced the amount of unwanted post-product, relative to non-MNase treated samples with or without the ExpiFectamine™ 293 Transfection Kit Enhancer (Thermo Fisher Scientific).

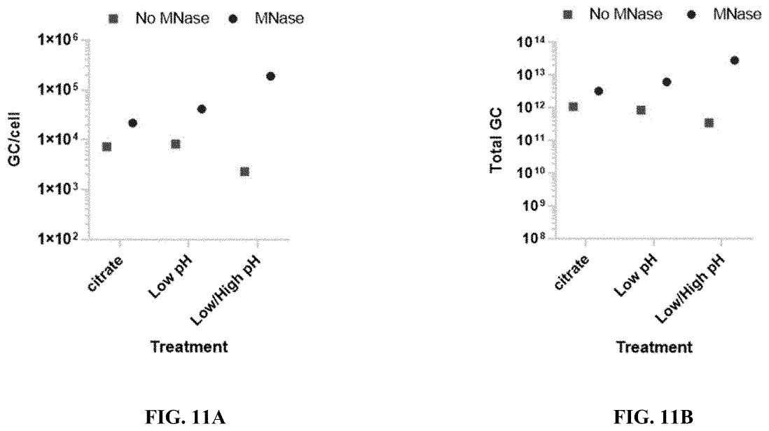

As shown in FIG. 11A and FIG. 11B, the increase in genome copies per cell (GC/cell) (FIG. 11A, Table 2), and total genome copies (FIG. 11B, Table 3) was consistently observed in MNase treated samples for each of the three different elution buffers (i.e., citrate, low pH, and low/high pH).

TABLE 2
Genome copies per cell (GC/Cell)

| | No MNase | MNase |
|---|---|---|
| Citrate | 7,250 | 21,800 |
| Low pH | 8,140 | 41,500 |
| Low/High pH | 2,300 | 190,000 |

TABLE 3
Total Genome Copies

| | No MNase | MNase |
|---|---|---|
| Citrate | 1.09e¹² | 3.27e¹² |
| Low pH | 8.48e¹¹ | 6.22e¹² |
| Low/High pH | 3.45e¹¹ | 2.84e¹³ |

Taken together, these data demonstrate that MNase treatment increased titers of AAV particles, and that the increase in titers of AAV particles was independent of the elution buffer.

### Example V

### MNase Treatment Increased Functional Titer of Product Fraction

This example establishes that MNase treatment increased the functional titer of the product fraction, as evidence by greater infectivity of the product fraction.

To evaluate the functional titer of the virus generated using MNase, target cells were transduced with viral particles expressing a red fluorescence protein collected from either the product fractions or the post-product fractions that had been treated with or without MNase. Functional titer was measured by determining whether the target cells exhibited any difference in red fluorescence protein expression as a surrogate for infectivity.

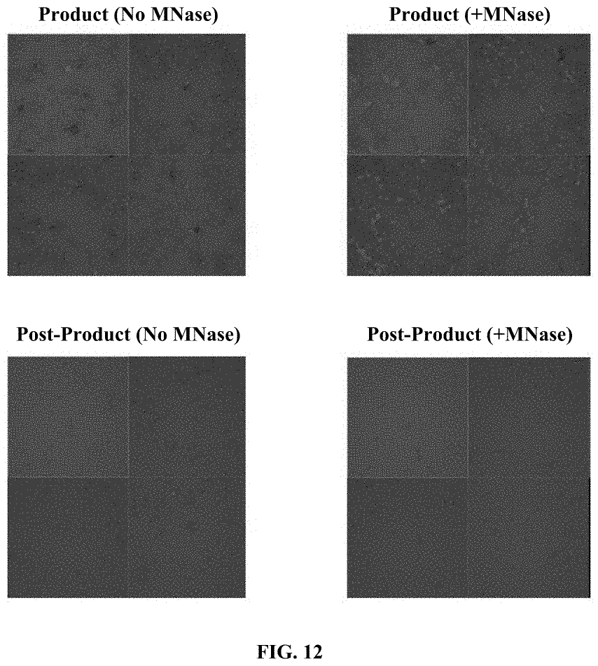

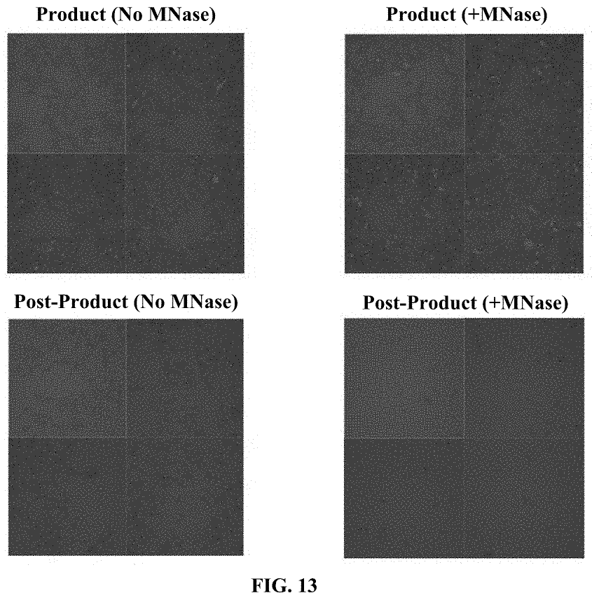

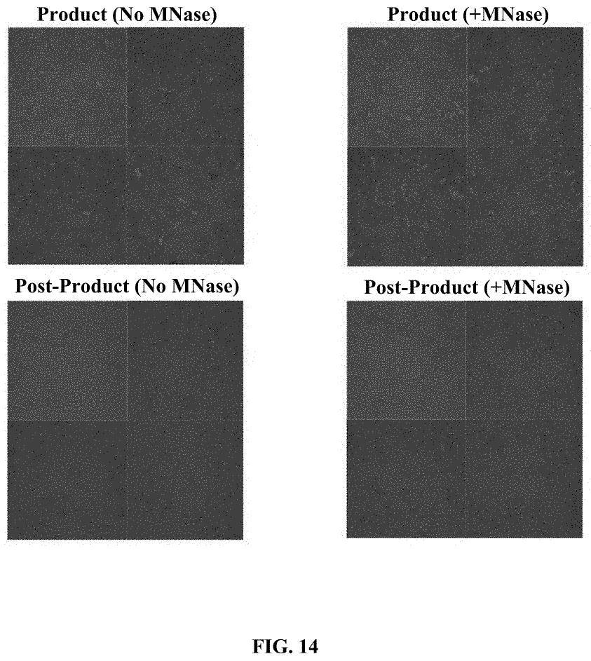

Briefly, samples were treated with or without MNase, eluted with high/low pH (FIG. 12), citrate (FIG. 13), or low pH (FIG. 14), and the product and post-product viral fractions were collected. The fractions were then contacted with cells, and the multiplicity of infection (MOI) was measured to determine functional titer levels.

The infectivity profile of the product fractions indicated that MNase treatment resulted in a greater MOI for each of the different elution buffers (Table 4). In contrast, the infectivity profile of the post-product fractions indicated that MNase treatment resulted in a decreased MOI for each of the different elution buffers.

TABLE 4
Infectivity Profile of Product Fractions

| | Product Fraction | | Post-product Fraction | |
|---|---|---|---|---|
| | No MNase | MNase | No MNase | MNase |
| Citrate | 3.8e⁶ | 9.9e⁶ | 7.1e⁵ | 6.9e⁴ |
| Low pH | 2.8e⁶ | 2.0e⁷ | 5.4e⁵ | 2.1e⁵ |
| Low/High pH | 1.4e⁶ | 2.0e⁷ | 2.0e⁵ | 8.9e⁴ |

These results indicated that MNase treatment increased the functional titer of the product fraction for each of the different elution buffers, but not the post-product fraction.

### Example VI

### High pH Wash Improved Purity of AAV

This example establishes that a high pH wash during affinity exchange chromatography of AAV was able to improve the purity of AAV.

To determine the optimal conditions for purifying AAV, the AKTA system was used and the samples were subjected to various wash and/or enzymatic treatment conditions during purification. The different purification conditions are summarized in Table 5.

TABLE 5
Purification Conditions

| | Affinity | | | | Anion Exchange |
|---|---|---|---|---|---|
| Condition | Wash pH | Benzonase ® | MNase | Elution | Elution |
| 1 | n/a | No | No | Citrate, pH 2.5 | 20 mM BTP, pH 10.2; |
| 2 | n/a | Yes | No | Citrate, pH 2.5 | 10 mM-200 mM NaCl |
| 3 | n/a | Yes | Yes | H₃PO₄, pH 1.5 | Linear Gradient |
| 4 | pH 9.5 | Yes | Yes | Citrate, pH 2.5 | |
| 5 | pH 10.2 | Yes | Yes | H₃PO₄, pH 1.5 | |
| 6 | pH 10.3 | Yes | Yes | H₃PO₄, pH 1.5 | |
| 7 | pH 10.3 | Yes | No | H₃PO₄, pH 1.5 | |
| 8 | pH 10.9 | Yes | Yes | H₃PO₄, pH 1.5 | |
| 9 | pH 10.4 | Yes | Yes | H₃PO₄, pH 1.5 | |
| 10 | pH 10.4 | Yes | Yes | H₃PO₄, pH 1.5 | 20 mM Tris pH 9.0; |
| | | | | | 0-300 mM (C₂H₅)NCl |
| | | | | | Linear Gradient |

BTP: Bis-Tris-Propane;
NaCl: sodium chloride;
H₃PO₄: phosphoric acid

Briefly, after bulk harvest and Benzonase® treatment, the crude samples were subjected to affinity chromatography. As indicated in Table 5, under specific conditions (i.e., conditions 4-10), the affinity column was washed with a high pH buffer that ranged from pH 9.5 to pH 10.9. In addition, as indicated in Table 5, the samples were either not treated with Benzonase® or MNase (condition 1), treated on-column with Benzonase® only (conditions 2, and 7) or treated on-column with Benzonase® and MNase (conditions 3-5, and 8-10). Elution from the affinity column was performed using citrate, pH 2.5 (conditions 1, 2, and 4), or phosphoric acid (H₃PO₄), pH 1.5 (conditions 3, and 5-10).

After the affinity chromatography purification, the samples were then polished by anion-exchange chromatography using CIM QA (quaternary amine) monolith columns. Thereafter, the samples were either eluted using a 10 mM-200 mM NaCl linear gradient and neutralized in 20 mM BTP, pH 10.2 (conditions 1-9) or eluted using a 0-300 mM (C₂H₅)NCl linear gradient and neutralized in 20 mM Tris pH 9.0 (condition 10).

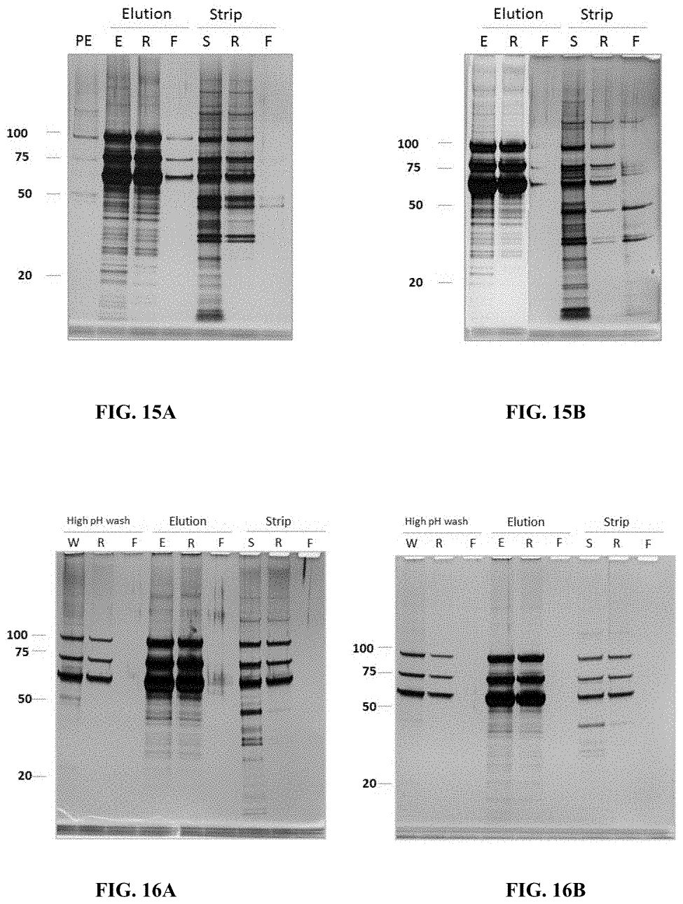

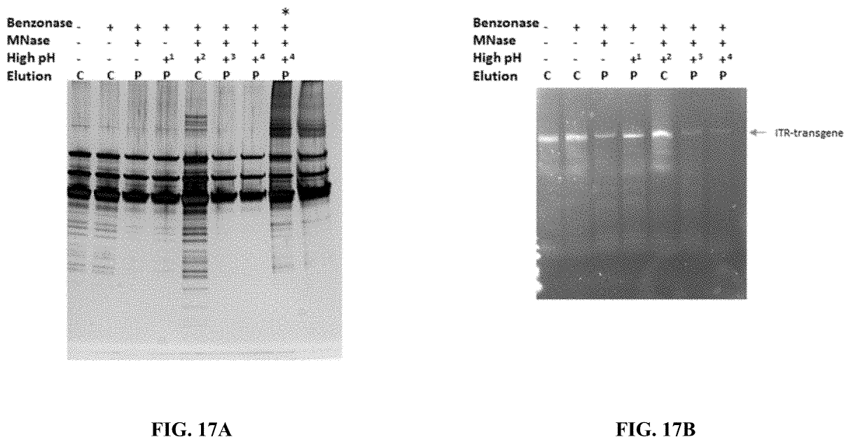

The purified samples were collected and subjected to sodium dodecyl sulfate-polyacrylamide gel electrophoresis (SDS-PAGE) followed by silver staining. In the conditions that did not include any enzymatic treatment (condition 1) or only included Benzonase® treatment (condition 2), the eluted AAV was found to have a large amount of impurities (FIG. 15A and FIG. 15B, respectively). For example, in addition to the three intense bands that correspond with VP1, VP2, and VP3, there were many other protein impurities detected by silver staining. In contrast, when the AAV sample was purified using a pH 9.5 wash during the affinity chromatography purification step (condition 4) there were appreciably less impurities in the elution fraction (FIG. 16A). A further improvement was observed using a pH 10.4 wash during the affinity chromatography purification step (FIG. 16B). However, washing the affinity column at pH 10.9 led to a large loss of virus. Comparison of the different purification conditions indicated that high pH wash conditions <pH 10.9, when combined with Benzonase® and MNase treatment, resulted in highly pure AAV samples (FIG. 17A). In addition, DNA agarose gel electrophoresis of the purified AAV samples indicated the presence of the ITR-transgene contained within the AAV.

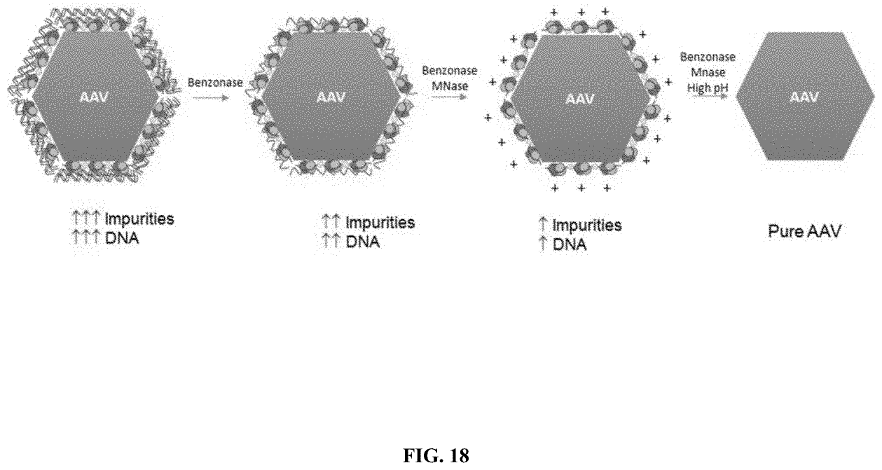

These results indicated that a high pH wash (˜pH 9.5-pH 10.4) during the affinity chromatography purification could improve the purity of the purified AAV (FIG. 18).

### Example VU

### Modification of Elution Conditions Improves Recovery of Virus

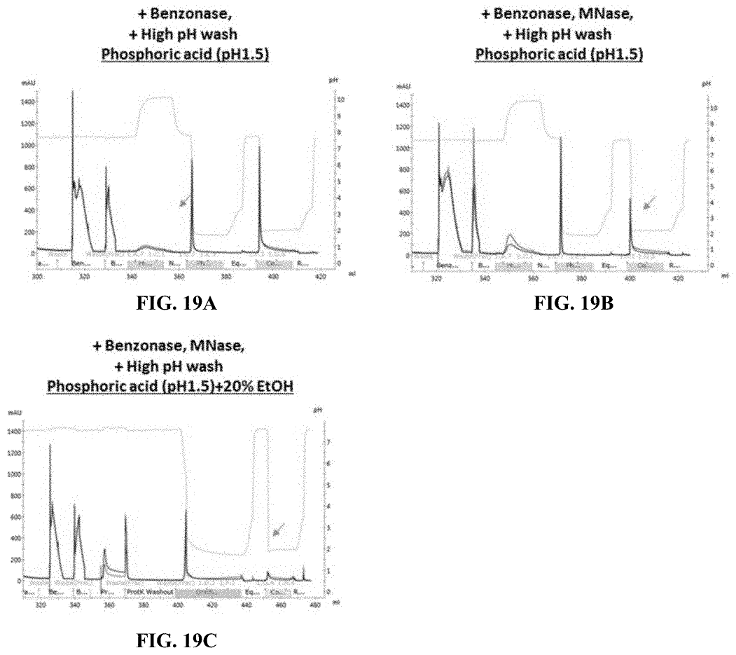

This example establishes that elution with a low pH buffer and ethanol was able to improve the purity of the purified AAV (FIG. 19).

To determine whether residual virus remaining on the anion-exchange chromatography column, samples were processed under three separate conditions, which included two different elution conditions. Briefly, the samples were treated with Benzonase® with or without MNase, subjected to affinity chromatography purification, and polished by anion-exchange chromatography, as described previously. The samples were then eluted using a low pH buffer (phosphoric acid, pH 1.5) with or without 20% ethanol.

The results indicated that in the samples that were treated with Benzonase® only (i.e., no MNase), washed with a high pH buffer, and eluted with phosphoric acid (pH 1.5), residual virus remained on the column after stripping, as highlighted by the indicated chromatography peak (see arrow) (FIG. 19A). The addition of MNase during the affinity chromatography purification step was able to reduce the residual virus during column strip (see arrow) (FIG. 19B). The elution of virus from the column was improved even further by the addition of 20% ethanol during elution (see arrow), as indicated by a near absence of the chromatography peak (see arrow) (FIG. 19C).

Taken together, these results indicated that the recovery of virus from the anion-exchange chromatography column could be further improved by the addition of ethanol during elution.

### Example VIH

### MNase Treatment Improved Removal of Impurities, as Measured by Dynamic Light Scattering (DLS) and Tm/Tagg

This example establishes that MNase treatment improved removal of impurities, as measured by Dynamic Light Scattering (DLS) and determination of the temperature at which protein aggregation Tagg aligns with the melting of the virus.

The thermal stability as a determinant of AAV serotype identity is known in the art, and can also be used to detect impurities in AAV particle samples. For examples, as the impurities as eliminated, the onset of protein aggregation (Tagg) aligns with the melting temperature (Tm) of virus.

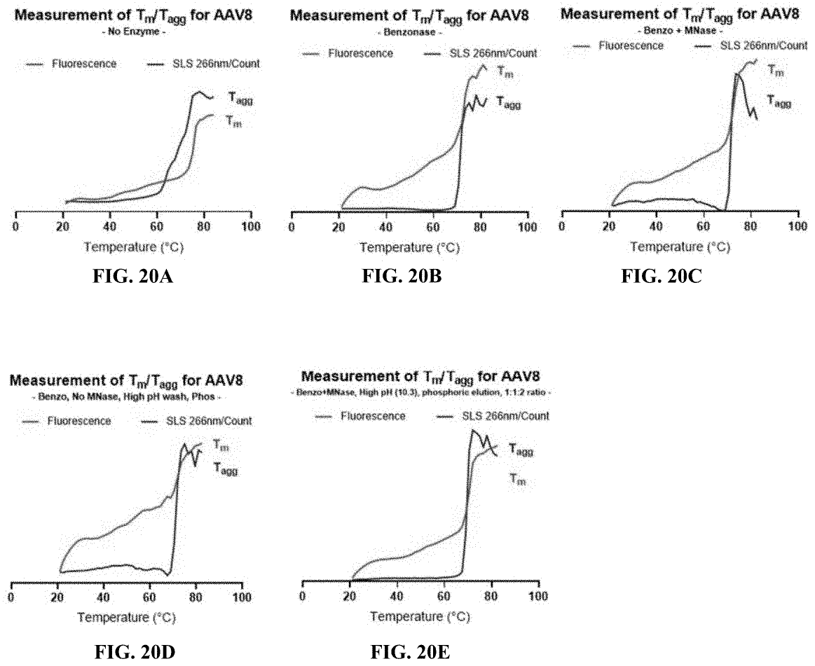

Consistent with previous results described above, the Dynamic Light Scattering (DLS) and Tm/Tagg assay was able to confirm that MNase improved the removal of impurities from the AAV particles. Specifically, in samples that were not treated with Benzonase® or MNase, the Tagg and Tm were found to differ by more than 10° C. (FIG. 20A). While the addition of Benzonase® improved the removal of impurities, Benzonase® and MNase together showed an improvement in the Tagg and Tm alignment (FIG. 20B and FIG. 20C).

Improvements in reducing the impurities were also observed in samples that were treated with MNase and eluted under the high pH conditions. Specifically, the Tagg and Tm were alignment for samples that were treated with Benzonase® and MNase and eluted with phosphoric acid (pH 10.3) (FIG. 20E) was enhanced, compared to samples that were treated with Benzonase® only, and eluted with phosphoric acid (pH 10.3) (FIG. 20D).

Taken together, these results confirmed that MNase treatment was able to reduce the amount of impurities in the AAV particle samples, and that MNase treatment combined with high pH conditions can reduce the amount of impurities even further.

### Example IX

### MNase Treatment During Purification Reduces Residual Host-Cell DNA

This example establishes that host-cell DNA can be significantly reduced by MNase treatment.

To determine whether any residual host-cell DNA was present in the AAV particle samples following anion-exchanges (AEX) purification, an AlphaLISA assay was performed to detect host-cell DNA as the analyte. The AlphaLISA bead-based technology relies on PerkinElmer's amplified luminescent proximity homogeneous assay and uses a luminescent oxygen-channeling chemistry to detect host-cell DNA (see Beaudet et al., Nat Methods 5, an8-an9 (2008)).

Analysis of residual host-cell DNA following AEX purification was measured by AlphaLISA in samples prepared under six different conditions: (1) purification without Benzonase® or MNase (“no enzyme”); (2) purification with Benzonase® and citrate elution (“B, citrate (affinity)”); (3) purification with Benzonase® and citrate elution (“B, citrate”); (4) purification with Benzonase®, MNase, and phosphoric acid elution (“B, M, Phos”); (5) purification with Benzonase®, high pH (pH 10.3) wash, and phosphoric acid elution (“B, pH 10.3, Phos”); and (6) purification with Benzonase®, MNase, and phosphoric acid elution (“B, M, pH 10.3, Phos”). A standard curve was generated to extrapolate the concentration levels of host-cell DNA present.

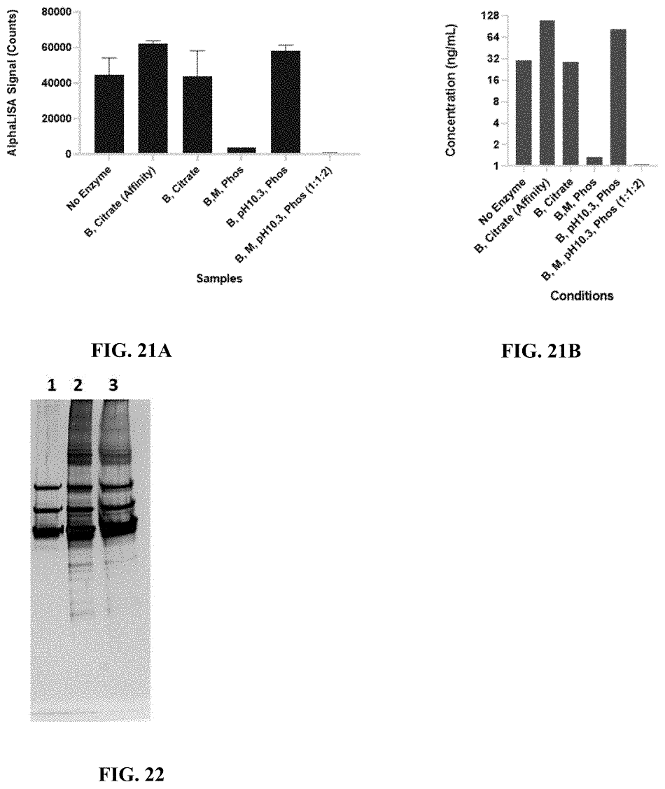

The results indicated that in the samples treated with MNase, the levels of host-cell DNA was significantly reduced (FIG. 21A and FIG. 20B). Notably, purification with Benzonase®, MNase, and phosphoric acid elution (“B, M, pH 10.3, Phos”) resulted in nearly undetectable levels of host-cell DNA (FIG. 21A and FIG. 21B).

These results indicated that MNase treatment significantly reduced host-cell DNA from AAV particle samples.

### Example X

### MNase Treatment Improves Purification Process

This example illustrates that the purification process provided herein represents an improvement over the state of the art.

Current methods of purification of AAV particles using anion-exchange and Benzonase® have been described (see, Wang C, et al. Mol Ther Methods Clin Dev. 2019; 15:257-263). However, MNase is not employed in such methods.

To determine whether the purification method described herein that includes MNase treatment can perform better than the current purification methods, the MNase treatment protocol (Protocol #1) was compared to the fractions obtained using the Wang et al. method (Protocol #2).

Briefly, purification according to Wang et al. was performed by affinity resin. Before loading to the column, the bulk AAV pool was treated with 50 U/mL Benzonase® at 37° C. for 1 h and clarified by centrifugation at 10,000×g for 15 min, followed by sequential filtrations through 1.2- and 0.45 mm filters. AAV was eluted from column with low-pH buffer. In parallel, a different set of samples were purified according to the methods described herein, which included MNase treatment.

Comparison between Protocol #1 and Protocol #2 revealed that MNase treatment was able to greatly reduce the amount of impurities present in the AAV particle samples, as measured by silver stain (FIG. 22).

Thus, this example demonstrates that the purification methods provided herein using MNase represent an improvement over the methods used for AAV purification.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method for purifying adeno-associated viral (AAV) particles, said method comprising:
(a) contacting a supernatant comprising AAV particles with a composition comprising a chromatin-DNA nuclease; and
(b) purifying the AAV particles.

2. The method of claim 1, wherein purifying comprises centrifugation, chromatography, filtration, or a combination thereof.

3. The method of claim 2, wherein centrifugation comprises density gradient centrifugation, ultracentrifugation, or a combination thereof.

4. The method of claim 2, wherein chromatography comprises affinity chromatography, ion exchange chromatography, size exclusion chromatography, hydrophobic interaction chromatography, or a combination thereof.

5. The method of claim 4, further comprising incubating the supernatant comprising AAV particles with a solid support for a sufficient amount of time to bind the AAV particles.

6. The method of claim 5, further comprising washing the solid support.

7. The method of claim 6, wherein the washing comprises a high pH buffer.

8. (canceled)

9. The method of claim 7, wherein the high pH buffer is between pH 9.0 and pH 11.

10. (canceled)

11. (canceled)

12. (canceled)

13. (canceled)

14. (canceled)

15. (canceled)

16. (canceled)

17. (canceled)

18. The method of claim 1, wherein the composition of step (a) further comprises Benzonase®.

19. The method of claim 1, wherein the incubation is for about 10 minutes to about 1 hour.

20. (canceled)

21. (canceled)

22. The method of claim 1, wherein the chromatin-DNA nuclease is micrococcal nuclease (MNase).

23. (canceled)

24. (canceled)

25. (canceled)

26. (canceled)

27. (canceled)

28. The method of claim 5, wherein the AAV particles are eluted from the solid support using a low pH buffer.

29. (canceled)

30. The method of claim 28, wherein the low pH buffer is less than about pH 3.0.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. The method of claim 22, wherein the purified AAV particles are substantially free of host-cell DNA, when compared to non-MNase contacted purified AAV particles.

47. The method of claim 46, wherein the host-cell DNA concentration is less than 2 ng/mL.

48. (canceled)

49. (canceled)

50. (canceled)

51. (canceled)

52. (canceled)

53. (canceled)

54. The method of claim 22, wherein the purified AAV particles have an increased viral titer, when compared to non-MNase contacted purified AAV particles.

55. (canceled)

56. The method of claim 54, wherein the viral titer comprises a functional titer.

57. (canceled)

58. (canceled)

59. (canceled)

60. (canceled)

61. (canceled)

62. (canceled)

63. (canceled)

64. (canceled)

65. (canceled)

66. (canceled)

67. The method of claim 22, wherein the purified AAV particles have a melting temperature (Tm) within less than about 10° C. of an aggregation temperature (Tagg), as measured by dynamic light scatter (DLS).

68. (canceled)

69. (canceled)

70. The method of claim 4, wherein the purified AAV particles comprise a full-to-empty capsid ratio of greater than about 50%.

71. (canceled)

72. (canceled)

73. (canceled)

74. (canceled)
